SEQUENCE LISTING (14 sequences)

SEQ ID NO: 1 - DNA, 731 bp, Pisum sativum
ttttttttttttttttatcatgggtgtctttgtttttgatgatgaatacgtttcaactgttgcaccacctaaactctacaaagctctcgcaaaagatgctgacgaaatcgtcccaaaggtgatcaaggaagcacaaggagtcgaaattatcgaaggaaatggaggtccaggaaccatcaagaagctatccattcttgaagatggaaaaaccaactatgtgctacacaaactagacgcagttgatgaagcaaactttggttacaactacagcttagtaggaggaccagggctacatgaaagtttagagaaagttgcattcgagacaattattttggctggttctgacggtggatccatcgttaagatatctgtgaaatatcacaccaaaggtgatgcagctctatctgatgcagttcgtgatgaaacaaaggccaaaggaactggacttatcaaggccatagaaggttacgttttggcaaatcctggttactaattagttgtataatcttctacttggtttggttttgttatgcgaataatgaatgaataaagtgttgtgatatggttttttaatttacatgtgtgaggctatgttgtcaagtgtgaactagtgtcggtttgagtgtggttttgggagaatttggttgggttgatgatgaggttttgtactatatgtatttctctaataaataatgcaaaagaaaagttcctagtaaaaaaaaaaaaaaaaa

SEQ ID NO: 2 - protein, 157 aa, Pisum sativum
MGVFVFDDEYVSTVAPPKLYKALAKDADEIVPKVIKEAQGVEIIEGNGGPGTIKKLSILEDGKTNYVLHKLDAVDEANFGYNYSLVGGPGLHESLEKVAFETIILAGSDGGSIVKISVKYHTKGDAALSDAVRDETKAKGTGLIKAIEGYVLANPGY

SEQ ID NO: 3 - DNA, 43 bp, Artificial
  Forward primer for PCR reactions using cDNA as template and pea ABR17
gtggtcgaagcttatgggtgtctttgtttttgatgatgaatac

SEQ ID NO: 4 - DNA, 43 bp, Artificial
  Reverse primer for PCR reactions using cDNA as template and pea ABR17
tatatagctcgagttagtaaccaggatttgccaaaacgtaacc

SEQ ID NO: 5 - DNA, 21 bp, Artificial
  Forward primer for PCR reaction using newly synthesized cDNA for detection of ABR17
ggtgatcaaggaagcacaagg

SEQ ID NO: 6 - DNA, 21 bp, Artificial
  Reverse primer for PCR reaction using newly synthesized cDNA for detection of ABR17
tttggcctttgtttcatcacg

SEQ ID NO: 7 - DNA, 20 bp, Artificial
  Forward primer for ABR17 transcript amplification
ccaggtccagacatagtaag

SEQ ID NO: 8 - DNA, 20 bp, Artificial
  Reverse primer for ABR17 transcript amplification
gtacaaagggcagggacgta

SEQ ID NO: 9 - DNA, 21 bp, Artificial
  Forward primer for quantitative real-time polymerase chain reaction (qRT-PCR) to determine level of ABR17
aaatggaggtccaggaaccat

SEQ ID NO: 10 - DNA, 25 bp, Artificial
  Reverse primer for quantitative real-time polymerase chain reaction (qRT-PCR) to determine level of ABR17
agcacatagttggtttttccatctt

SEQ ID NO: 11 - DNA, 17 bp, Artificial
  Probe sequence for quantitative real-time polymerase chain reaction (qRT-PCR) to determine level of ABR17
agaagctatccattctt

SEQ ID NO: 12 - DNA, 19 bp, Artificial
  Forward primer for the amplification of the actin gene
gccattcaggccgttcttt

SEQ ID NO: 13 - DNA, 21 bp, Artificial
  Reverse primer for the amplification of the actin gene
atcgagcacaataccggttgt

SEQ ID NO: 14 - DNA, 16 bp, Artificial
  Probe sequence for the ampilfication of the actin gene
tctatgccagtggtcg

(19) United States
(12) Patent Application Publication
(10) Pub. No.: US 2008/0005810 A1
(43) Pub. Date: Jan. 3, 2008

(54) METHOD OF CONFERRING MULTIPLE STRESS TOLERANCE AND EARLY FLOWERING IN PLANTS

(75) Inventors: Nataraj N. KAV, Edmonton (CA); Sanjeeva SRIVASTAVA, Edmonton (CA); Salehuzzaman SHAH, Edmonton (CA)

Correspondence Address: EDWARD YOO C/O BENNETT JONES, 1000 ATCO CENTRE, 10035 - 105 STREET, EDMONTON, ALBERTA, AB, T5J3T2, CA

(73) Assignee: THE GOVERNORS OF THE UNIVERSITY OF ALBERTA, Edmonton (CA)

(21) Appl. No.: 11/750,797

(22) Filed: May 18, 2007

Related U.S. Application Data
(60) Provisional application No. 60/747,646, filed on May 18, 2006.

(51) Int. Cl. A01H 1/00; A01H 5/00; C12N 15/00

(52) U.S. Cl. 800/278; 435/320.1; 800/298; 800/306

(57) ABSTRACT

Transgenic plants are more tolerant to environmental stresses than untransformed plants. The pea ABR17 (Abscisic acid responsive 17) is used to enhance germination of plants such as Arabidopsis sp. and Brassica sp. while under multiple abiotic stresses, and to enhance the tolerance of these plants to these stresses. Three independently derived Arabidopsis transgenic lines, containing ABR17, germinated better in the presence of salt, cold temperature or both. The transgenic plants also exhibited enhanced tolerance to freezing temperature or extreme heat. Furthermore, the transgenic plants demonstrated early flowering even under normal, non-stressed conditions.

### CROSS REFERENCE TO RELATED APPLICATIONS

This application is a divisional application of U.S. Provisional Patent Application 60/747,646 filed on May 18, 2006 entitled “Method of Conferring Multiple StressTolerance to Plants”, the contents of which are incorporated herein by reference.

### FIELD OF THE INVENTION

The present invention relates to a method of enhancing plant tolerance to abiotic stresses and/or promoting early flowering in plants, by introducing genes that confer stress tolerance or early flowering into plant cells, seeds or plants.

### BACKGROUND

Plants encounter a wide range of environmental stresses including drought, salinity, temperature extremes, flooding and ultraviolet radiation, which severely limit crop productivity worldwide (Boyer 1982). Abiotic stresses can occur simultaneously and could affect multiple stages of plant growth and development (Chinnusamy et al., 2004)_leading to serious morphological, physiological, biochemical and molecular changes and can cause concomitant reduction in the average yield of most major crops by more than 50% (Boyer 1982; Bray et al., 2000; Wang et al., 2003). Soil salinity alone can lead to a significant drop in the yield, affecting as much as 7% of the world's arable land (Hasegawa et al., 2000; Zhu 2003). Furthermore, increased salinization is expected to reduce agricultural land by an estimated 30% by the year 2025 and up to 50% by the middle of the 21st century (Wang et al., 2003). In the extreme cold conditions of the Canadian Prairies as well as in other parts of the world, successful growth of many crops is based on their ability to germinate and survive better in the cold temperature (Coursolle et al., 1998).

The phytohormone abscisic acid (ABA) plays a crucial role in response to environmental stresses such as desiccation, salinity and cold and it is also involved in regulating events such as dormancy and maturation during late seed development (McCarty 1995; Leung and Giraudat 1998). Many ABA-inducible genes share the cis-regulatory elements (i.e., ABA-responsive elements; ABREs; Guiltinan et al., 1990) and have been demonstrated to be involved in the regulation of plant stress responses. Constitutive expression of ABF3 in Arabidopsis (Kang et al., 2002) and rice (Oh et al., 2005) enhanced tolerance to drought stress as well as tolerance to multiple stresses in Arabidopsis (Kim et al., 2004).

Several gene products homologous to ABA-responsive proteins (also known as dehydrins and late embryogenesis abundant or LEA proteins) have been identified in different plant systems (Skriver and Mundy 1990; Close et al., 1993). It has been suggested that dehydrins (members of LEA proteins) and small heat shock proteins (sHSPs) may protect cells from the deleterious effects of dehydration (Pneuli et al., 2002). Expression of the HVA1 gene of barley, which encodes a LEA protein, conferred tolerance to salinity and water-deficit stresses (Xu et al., 1996), a tomato Le25 gene in yeast increased freezing and salinity tolerance (Imai et al., 1996), and a wheat chloroplast LEA-like protein (WCS 19) in Arabidopsis (Ndong et al., 2002) resulted in increased freezing tolerance.

Enhancing plant tolerance to abiotic stresses by introducing genes is a desirable method for many crop plants. For example, the over-expression of vacuolar Na+/H+ antiporter AtNHX1 promoted growth and development in saline conditions (up to 200 mM NaCl) in Arabidopsis (Apse et al., 1999) and in B. napus (Zhang et al., 2001). With respect to tolerance to cold temperatures, transgenic plants over-expressing various transcription factors have been demonstrated to have a higher tolerance. For example, in Arabidopsis, significant improvement of freezing stress tolerance was demonstrated by the over-expression of the transcription factor CBF1 (Jaglo-Ottosen et al., 1998); enhancement of drought and freezing tolerance by CBF4 (Haake et al., 2002) and increased tolerance to freezing, water and salinity stress by over-expression of DREB1A gene (Kasuga et al., 1999), whereas transgenic rice over-expressing CBF3 demonstrated elevated tolerance to drought and salinity but very low freezing tolerance (Oh et al., 2005). It appears, therefore, that these transcription factors are able to enhance tolerance to a variety of stresses however such enhancement may be species-specific (Oh et al., 2005).

The availability of high throughput genomics and proteomics technologies enables scientists to conduct a comprehensive study of the genome, the transcriptome and the proteome under different abiotic and biotic stress conditions (Ramonell and Somerville 2002; Agrawal et al., 2005). An analysis of salinity-induced changes in pea root proteome demonstrated an increase in the levels of several members of group 10 family of pathogenesis-related proteins (PR 10; Kav et al., 2004), including PR 10.1 and the ABA-responsive protein ABR17 (PR 10.4), in response to stress. The PR proteins are part of a multicomponent defense response in many plants that respond to various abiotic and biotic stresses (Walter et al., 1990). They are grouped into 14 different families based on their serological relations, homology at the nucleotide/amino acid sequence level and similarities in biological functions (van Loon etal., 1994; 1999).

Proteins belonging to the PR 10 family have been detected in a variety of angiosperms, monocots as well as dicot plants (Biesiadka et al., 2002). PR 10 proteins are small (15-18 kDa), acidic and intracellular, unlike other PR proteins that are extracellular (Walter et al., 1990; van Loon et al., 1994). These intracellular PR (IPR) proteins were first described in cultured parsley cells upon elicitor treatment (Somssich et al., 1988) and as described earlier, have subsequently been detected in many species. PR 10 genes are known to be induced by pathogens (Fristensky et al. 1985; McGee et al. 2001; Borsics and Lados 2002) as well as other stresses including salinity, drought, wounding and darkness (Osmark et al. 1998; Hashimoto et al., 2004; Kav et al., 2004). In addition to the induction by stimulus, PR 10 proteins occur in high concentrations in roots, flowers and pollen (Biesiadka et al., 2002 and references therein). These observations suggest that in addition to their role(s) in plant stress response, they could play an important role during the normal growth and development of plants (Wu et al., 2003).

The pea ABA-responsive protein ABR17 is similar to pea disease resistance response proteins; it is produced late in seed development, and is induced by exogenous application of ABA (Iturriaga et al., 1994; Colditz et al., 2004). Many proteins with significant homology to the pea ABA-responsive protein include an IPR protein from bean (Walter et al., 1990), garden pea (Fristensky et al., 1988), parsley (Somssich et al., 1988), major birch pollen allergen Bet v1 (Breiteneder et al., 1989), potato (Constabel and Brisson, 1992), and SAM22 from soybean (Crowell et al., 1992). Several gene products homologous to ABR17, proteins also known as dehydrins and late embryogenesis abundant (LEA) related proteins, have been identified in different plant systems (Skriver and Mundy, 1990; Close et al., 1993; Goday et al., 1994). Srivastava et al., (2004) reported that the constitutive expression of a PR 10 (PR 10.1) gene from pea in Brassica napus ameliorates the effects of salinity stress during germination and early seedling growth.

Despite the identification of proteins with significant similarities to ABR17 (and other PR 10) in many species, a direct role for these proteins in mediating plant responses to abiotic stresses has not been demonstrated.

### SUMMARY OF THE INVENTION

In one aspect, the invention described herein is directed to transgenic plants that are more tolerant to environmental stresses than untransformed plants. Demonstrated herein is the use of the pea ABR17 (Abscisic acid responsive 17) to enhance germination of plants such as Arabidopsis sp. and Brassica sp. while under one or more stresses, and to enhance the tolerance of these plants to these stresses. Three independently derived Arabidopsis transgenic lines, containing ABR17, germinated better in the presence of salt, cold temperature or both. Furthermore, the transgenic plants also exhibited enhanced tolerance to freezing temperature or extreme heat, suggesting the potential utility of the ABR17 gene to engineer multiple stress tolerance. Two independently derived transgenic Brassica lines germinated better than the wild-type strain, in the presence of salt, salt and cold temperature, or darkness and salt.

In one aspect, this invention comprises an expression vector for transformation of a plant cell comprising:
(a) a nucleic acid sequence encoding abscisic responsive protein 17 (ABR 17) or a biologically active portion or variant thereof;
(b) regulatory elements operatively linked to the nucleic acid sequence encoding ABR17 or a biologically active portion or variant thereof, such that the nucleic acid sequence is expressed in the plant cell,
   wherein said expression results in expression of the protein encoded by the polynucleotide, in said plant cell. In one embodiment, the nucleic acid sequence in the vector encodes the amino acid sequence of SEQ ID NO:2. In one embodiment, the nucleic acid sequence comprises SEQ ID NO:1 or a variant or portion thereof, for example, nucleotides 20 to 493 of SEQ ID NO:1.

In another aspect, the invention comprises a transgenic plant cell transformed with the expression vector. In one embodiment, the transgenic plant cell is transformed with an expression vector comprising a nucleic acid sequence that encodes the amino acid sequence of SEQ ID NO: 2.

In another aspect, the invention comprises a transgenic plant seed comprising a transgenic plant cell transformed by the expression vector, and transgenic plants grown from a transgenic plant cell that is transformed with the expression vector. In one embodiment, the transgenic plant is grown from a plant cell or seed that is transformed with an expression vector comprising a nucleic acid sequence that encodes the amino acid sequence of SEQ ID NO: 2. The transgenic plant may be monocot or a dicot plant, for example, a transgenic Arabidopsis sp. or a transgenic Brassica sp.

In another aspect, the invention comprises a method of increasing the tolerance of a plant to at least one environmental stress, comprising the steps of:
(a) transfecting cells of said plant with a nucleic acid sequence encoding ABR17,
(b) selecting and maintaining from said cells a transgenic cell line that expresses a protein encoded by said polynucleotide, and
(c) producing a plant from the transgenic cell line;
(d) wherein the increased tolerance to the at least one environmental stress is demonstrated by one or more of enhanced germination, greater rate of flowering, earlier flowering, greater plant height, increased root length, increased shoot length, overall plant health as compared to a control plant.

In one embodiment, the nucleic acid sequence encoding ABR17 encodes an amino acid sequence which comprises SEQ ID NO: 2. In one embodiment, the nucleic acid sequence encoding ABR17 comprises SEQ ID NO:1. In one embodiment, the cells used in step (a) are from Arabidopsis sp or the cells used in step (a) are from Brassica sp.

In another aspect, the invention may comprise a method of promoting early flowering in a plant under normal conditions, or under one or more abiotic stresses, comprising the steps of:
(a) transfecting cells of said plant with a nucleic acid sequence encoding ABR17,
(b) selecting and maintaining from said cells a transgenic cell line that expresses a protein encoded by said polynucleotide, and
(c) producing a plant from the transgenic cell line.

### BRIEF DESCRIPTION OF THE DRAWINGS

FIG. 1: Expression of ABR17 cDNA in transgenic A. thaliana. (a) RT-PCR analysis of ABR17 and 18s RNA expression demonstrating the presence of ABR17 transcript in the 3 transgenic lines (6.9, 14.9, 25.20) and its absence in the wild type (WT) as well as the presence of 18s transcript throughout. (b) Western blot analysis of protein extracts (from 2 week-old seedlings) demonstrating the presence of a unique band corresponding to the molecular weight of ABR17 in the three transgenic lines.

FIG. 2: (a-e) demonstrate the effects of NaCl and low temperature on the germination of wild type (WT) seeds and seeds from three transgenic Arabidopsis lines (6.9, 14.9 and 25.20). Room temperature and 75 mM NaCl (a) or 150 mM NaCl (b), or 10° C. and no NaCl (c), 75 mM NaCl (d) or 150 mM NaCl (e). Experiments were repeated three times (n=42) and the bars indicate the standard errors.

FIG. 3: Effects of salinity on seedlings germinated and grown at room temperature. Morphology of seedlings germinated after (a) 1 and (b) 2 weeks of growth at 0, 75 and 150 mM NaCl; (c) total chlorophyll and (d) carotenoid levels following two weeks of growth at 0 and 75 mM NaCl.

FIG. 4: Effects of salinity on the seedlings germinated and grown at 10° C. (a) and (b) are panels showing the growth of wild-type (WT) seedlings and seedlings of three transgenic Arabidopsis lines (6.9, 14.9 and 25.20), respectively, after two weeks (a) and three weeks (b) of growth at 0, 75 and 150 mM NaCl.

FIG. 5: Freezing-tolerance of ABR17 transgenic plants. (a-d) are panels showing the growth of seedlings from wild-type (WT) and three transgenic Arabidopsis lines (6.9, 14.9 and 25.20) lines carrying the ABR17 gene, one day after exposure to cold stress (−5° C.) for 4 h. Experiments were repeated three times (n=28).

FIG. 6: Heat-tolerance of ABR17 transgenic plants. (a-d) are panels showing the growth of wild-type (WT) seedlings and seedling from three transgenic Arabidopsis lines (6.9, 14.9 and 25.20) carrying the ABR17 gene, one day after exposure to heat stress (48° C.) for 2 h. Experiments were repeated three times (n=28).

FIG. 7: Images of two-dimensional gels of protein extracted from wild type and transgenic plants. (a) representative images of whole gels and (b) a closer view of changes in the intensities of the spots selected for identification.

FIG. 8. Expression of ABR17 cDNA in transgenic B. napus. (A) Analysis of ABR17 and 18s RNA expression by RT-PCR. The presence of ABR17 transcript in the transgenic lines (3.15 and 9.5) and its absence in the wild type (WT) as well as 18s RNA transcript in both lines is visualized by staining with ethidium bromide. (B) Analysis of protein extracts from two-week old seedlings by Western blot. The presence of a unique, immunoreactive band in both of the transgenic lines (3.15 and 9.5) at the expected molecular weight indicates the presence of ABR17.

FIG. 9. Appearance of WT and ABR17 transgenic B. napus seedlings and plants. Appearance of (A) 7-day, (B) 14 day- and 37 day-old seedlings (C and E). The appearance of 42-day old plants demonstrating early flowering in the transgenic lines is shown in panels D and F.

FIG. 10. Effect of NaCl on germination, root length and shoot length of WT and ABR17 transgenic B. napus seedlings. (A) Effect of 275 mM NaCl on germination, (B) root length (cm), and (C) shoot length (cm) of 7 day-old seedlings.

FIG. 11. Effect of low temperature and NaCl on the germination of WT and ABR17 transgenic B. napus seedlings. (A) The effect of 5° C., (B) 10° C. and (C) 10° C. plus 75 mM NaCl on the germination of the WT and transgenic lines 3.15 and 9.5.

FIG. 12. Flowering and height of ABR17 transgenic and WT B. napus adult plants 42 days after planting (DAP). (A) First day of flowering, (B) rate of flowering, (C) plant height of ABR17 transgenic and WT B. napus adult plants and (D) the relative expression of ABR17 in the transgenic lines (3.15 and 9.5).

### DETAILED DESCRIPTION OF PREFERRED EMBODIMENTS

Throughout this disclosure, various publications, patents and published patent specifications are referenced. Where permissible, the disclosures of these publications, patents and published patent specifications are hereby incorporated by reference in their entirety into the present disclosure to more fully describe the state of the art. Unless otherwise indicated, the disclosure encompasses conventional techniques of molecular biology, microbiology, cell biology and recombinant DNA, which are within the skill of the art- see e.g., Maniatis (1989); Sambrook and Russell (2001); Ausubel et al. (1987) and (2005).

Unless otherwise noted, technical terms are used according to conventional usage. Definitions of common terms in molecular biology may be found in Lewin (2000); Kendrew et al. (1994); Meyers (1995); Ausubel et al. (1987) and (2005); Sambrook and Russell (2001).

The results described here demonstrate, surprisingly and unexpectedly, that: (1) constitutive expression of pea (Pisum sativum) ABR17 gene in Arabidopsis enhances germination and early seedling growth in the presence of salt or cold or when both stresses are combined, (2) two week-old transgenic Arabidopsis plants exhibit increased tolerance to multiple environmental stresses (3) transgenic Brassica comprising pea ABR17 gene have enhanced germination and early seedling growth in the presence of cold, salt and cold, or salt and darkness, and (4) transgenic Brassica comprising pea ABR17 exhibited early flowering under normal conditions. Therefore, the applicants demonstrate that pea ABR17 is capable of protecting plants from multiple abiotic stresses, particularly cold and salinity. This protection is evident during the germination of the transgenic seeds in the presence of salt or a combination of salt and cold and also when older plants, such as seedlings, are subjected to freezing or heat stresses. Furthermore, pea ABR17 is capable of promoting early flowering, a trait which is very crucial for early maturity of Brassica sp. in short growing seasons.

The proteome of a transgenic A. thaliana line 6.9 expressing pea ABR17 gene, when compared to its wild type counterpart, demonstrates that the levels of several proteins involved in photosynthesis or primary metabolic pathways (Table 2) were significantly (P<0.01) affected in the transgenic Arabidopsis line. The proteins showing increased levels in the transgenic line are: (a) PSI-E (spot 2; Table 2), a component of photosystem I (PSI); (b) oxygen-evolving protein, PSBO-2/PSBO2 (spot 12; Table 2), which showed ˜3 fold up-regulation; (c) rubisco activase, which showed ˜2-fold increase and (d) glycine-rich RNA-binding proteins (GR-RBP; spots 16 and 17; Table 2). Therefore, it is possible that the increase of one or more of these proteins in ABR17 transgenic lines may contribute to the observed increase in stress tolerance.

The level of another enzyme involved in primary metabolism, phosphopyruvate hydratase (spot 14; Table 2) also known as enolase (2-phospho-D-glycerate hydrolase) was decreased in the transgenic line. The observed reduction in enolase level in the ABR17 transgenic A. thaliana line may be important for its tolerance to cold temperature stress.

To facilitate understanding of the invention, a number of terms are defined below.

“Stress” refers to a factor that externally causes a change in the growth of plants. “Environmental stress” and “environmental stresses” refer to a stress, or stresses as the case may be, provided by a change in an external environment, including salt concentration, high osmotic pressure, drying, high temperature, low temperature, intense light, air pollution, and the like.

“Expression” refers to transcription or translation, or both, as context requires.

The terms “modified”, “mutant” or “variant” are used interchangeably herein, and refer to: (a) a nucleotide sequence in which one or more nucleotides have been added or deleted, or substituted with different nucleotides or modified bases (e.g., inosine, methylcytosine) or to (b) a protein, peptide or polypeptide in which one or more amino acids have been added or deleted, or substituted with a different amino acid. A variant may be naturally occurring, or may be created experimentally by one of skill in the art. A variant of SEQ ID NO:1 or SEQ ID NO: 2 may be a protein, peptide, polypeptide or polynucleotide that differs (i.e., an addition, deletion or substitution) in one or more amino acids or nucleotides from the sequence presented as SEQ ID NO:1 or SEQ ID NO: 2.

A variant protein may include analogues or homologues having similar or identical biologic activity native in other plant species. The present invention is intended to encompass all homologues, paralogues and analogues of acyl CoA binding protein, and biologically active portions and variants thereof.

In this regard, it is well understood in the art that certain alterations inclusive of mutations, additions, deletions and substitutions can be made to a reference nucleic acid or protein, to result in a modified, mutant or variant nucleic acid or protein that retains a particular biological function or activity, or perhaps displays an altered but nevertheless useful activity. Some deletions, insertions and substitutions will not produce radical changes in the characteristics of the protein having the sequence SEQ ID NO:2 or in the nucleic acid represented by SEQ ID NO:1. However, while it may be difficult to predict the exact effect of the substitution, deletion or insertion in advance of doing so, one skilled in the art will appreciate that the effect can be evaluated by routine screening assays. For example whether a variant of the protein having the sequence SEQ ID NO:2 enhances germination, confers stress tolerance or promotes early flowering, can be determined by following the methods disclosed in the Examples disclosed herein. Modifications of protein properties such as redox or thermal stability, hydrophobicity, susceptibility to proteolytic degradation, or the tendency to aggregate with carriers or into multimers may be assayed by methods well known to one of skill in the art.

Variants may be created experimentally using random mutagenesis, oligonucleotide-mediated (or site-directed) mutagenesis, PCR mutagenesis and cassette mutagenesis. Oligonucleotide-mediated mutagenesis is well known in the art as, for example, described by Adelman (1983) using vectors that are either derived from bacteriophage M13, or that contain a single-stranded phage origin of replication as described by Viera et al. (1987). Production of single-stranded template is described, for example, in Sambrook (2001). Alternatively, the single-stranded template may be generated by denaturing double-stranded plasmid (or other DNA) using standard techniques.

Alternatively, linker-scanning mutagenesis of DNA may be used to introduce clusters of point mutations throughout a sequence of interest that has been cloned into a plasmid vector. For example, reference may be made to Ausubel et al. (1987) and (2005). Region-specific mutagenesis and directed mutagenesis using PCR may also be employed to construct variants according to the invention. In this regard, reference may be made, for example, to Ausubel et al. (1987) and (2005). With regard to random mutagenesis, methods include incorporation of dNTP analogs (Zaccolo et al., (1996)) and PCR-based random mutagenesis such as described in Stemmer (1994) and Shafikhani et al. (1997).

A variant protein, polypeptide or peptide may, for example, have an amino acid sequence substantially identical to the specific sequences disclosed herein. By the term “substantially identical” it is meant that two polypeptide sequences preferably are at least 70% identical, and more preferably are at least 85% identical and most preferably at least 95% identical, for example 96%, 97%, 98% or 99% identical. In order to determine the percentage of identity between two polypeptide sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990: 215: 403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

The term “biologically active” when made in reference to a variant or portion of an ABR17 protein, refers to a protein, polypeptide or peptide possessing the ability to enhance germination or confer stress tolerance to a plant species that is transfected with a nucleic acid encoding that protein, polypeptide or peptide. Whether germination is enhanced in, whether the stress tolerance is conferred to transgenic plants, or whether early flowering is promoted may be determined, for example, by using the methods disclosed herein.

The term “portion” when used in reference to a protein refers to fragments of that protein. The fragments may range in size from four amino acid residues to the entire amino acid sequence of the protein, minus one amino acid.

An “expression vector” refers to a recombinant DNA molecule containing the appropriate control nucleotide sequences (e.g., promoters, enhancers, repressors, operator sequences and ribosome binding sites) necessary for the expression of an operably linked nucleotide sequence in a particular host cell. By “operably linked/linking” or “in operable combination” is meant that the nucleotide sequence is positioned relative to the control nucleotide sequences to initiate, regulate or otherwise direct transcription and/or the synthesis of the desired protein molecule.

As used herein, an “ABR17 protein” is a pea abscisic acid-responsive protein and includes a protein having the amino acid sequence of SEQ ID NO:2, and includes biologically active portions or variants thereof.

The ABR17 mRNA has the following nucleotide sequence (Accession # Z15128) [SEQ ID NO:1] where the coding region begins at nucleotide 20 and ends at nucleotide 493:

tttttttttt ttttttatca tgggtgtctt tgtttttgat
gatgaatacg tttcaactgt tgcaccacct aaactctaca
aagctctcgc aaaagatgct gacgaaatcg tcccaaaggt
gatcaaggaa gcacaaggag tcgaaattat cgaaggaaat
ggaggtccag gaaccatcaa gaagctatcc attcttgaag
atggaaaaac caactatgtg ctacacaaac tagacgcagt
tgatgaagca aactttggtt acaactacag cttagtagga
ggaccagggc tacatgaaag tttagagaaa gttgcattcg
agacaattat tttggctggt tctgacggtg gatccatcgt
taagatatct gtgaaatatc acaccaaagg tgatgcagct
ctatctgatg cagttcgtga tgaaacaaag gccaaaggaa
ctggacttat caaggccata gaaggttacg ttttggcaaa
tcctggttac taattagttg tataatcttc tacttggttt
ggttttgtta tgcgaataat gaatgaataa agtgttgtga
tatggttttt taatttacat gtgtgaggct atgttgtcaa
gtgtgaacta gtgtcggttt gagtgtggtt ttgggagaat
ttggttgggt tgatgatgag gttttgtact atatgtattt
ctctaataaa taatgcaaaa gaaaagttcc tagtaaaaaa
aaaaaaaaaa a

The ABR17 protein has the following amino acid sequence [SEQ ID NO:2]:

MGVFVFDDEYVSTVAPPKLYKALAKDADEIVPKVIKEAQGVEIIEGNGGP
GTIKKLSILEDGKTNYVLHKLDAVDEANFGYNYSLVGGPGLHESLEKVAF
ETIILAGSDGGSIVKISVKYHTKGDAALSDAVRDETKAKGTGLIKAIEGY
VLANPGY

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The term “nucleotide” refers to a ribonucleotide or a deoxyribonucleotide. “Nucleic acid” refers to a polymer of nucleotides and may be single- or double-stranded. “Polynucleotide” refers to a nucleic acid that is twelve (12) or more nucleotides in length. A nucleic sequence “encodes” or “codes for” a protein, polypeptide or peptide if the nucleotide sequence can be translated to the amino acid sequence of the protein, polypeptide or peptide.

The term “nucleic acid sequence encoding ABR17” refers to any and all nucleic acid sequences encoding an ABR17 protein. Nucleic acid sequences encoding an ABR17 protein further include any and all nucleic acid sequences which (i) encode polypeptides that are substantially identical to a ABR17 protein; or (ii) hybridize to any nucleic acid sequences encoding an ABR17 protein under at least moderately stringent hybridization conditions or which would hybridize thereto under at least moderately stringent conditions but for the use of synonymous codons.

By the phrase “at least moderately stringent hybridization conditions”, it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tₘ, which in sodium containing buffers is a function of the sodium ion concentration and temperature (Tₘ=81.5° C.−16.6 (Log₁₀ [Na⁺])+0.41(%(G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tₘ, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5× Denhardt's solution/1.0% SDS at Tₘ (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

Preparation of Recombinant Expression Vectors Comprising Chimeric Nucleic Acid Sequences Encoding ABR17 and a Nucleic Acid Sequence Capable of Controlling Expression in a Plant Cell

The term “chimeric” as used herein in the context of nucleic acid sequences refers to at least two linked nucleic acid sequences which are not naturally linked. Chimeric nucleic acid sequences include linked nucleic acid sequences of different natural origins. For example a nucleic acid sequence constituting a plant promoter linked to a nucleic acid sequence encoding an ABR17 is considered chimeric. Chimeric nucleic acid sequences also may comprise nucleic acid sequences of the same natural origin, provided they are not naturally linked. For example a nucleic acid sequence constituting a promoter obtained from a particular cell-type may be linked to a nucleic acid sequence encoding a polypeptide obtained from that same cell-type, but not normally linked to the nucleic acid sequence constituting the promoter. Chimeric nucleic acid sequences also include nucleic acid sequences comprising any naturally occurring nucleic acid sequence linked to any non-naturally occurring nucleic acid sequence.

The nucleic acid sequences encoding an ABR17 protein that may be used in accordance with the methods and compositions provided herein may be any nucleic acid sequence encoding an ABR17 protein.

Alterations to the nucleic acid sequence encoding ABR17 to prepare ABR17 analogs may be made using a variety of nucleic acid modification techniques known to those skilled in the art, including for example site directed mutagenesis, targeted mutagenesis, random mutagenesis, the addition of organic solvents, gene shuffling or a combination of these and other techniques known to those of skill in the art (Shraishi et al., 1988, Arch. Biochem. Biophys, 358: 104-115; Galkin et al., 1997, Protein Eng. 10: 687-690; Carugo et al., 1997, Proteins 28: 10-28; Hurley et al., 1996, Biochem, 35: 5670-5678; Holmberg et al., 1999, Protein Eng. 12: 851-856).

In accordance herewith the nucleic acid sequence encoding ABR17 is linked to a nucleic acid sequence capable of controlling expression of the ABR17 protein in a plant cell. Accordingly, the present invention also comprises a nucleic acid sequence encoding ABR17 linked to a promoter capable of controlling expression in a plant cell. Nucleic acid sequences capable of controlling expression in plant cells that may be used herein include any plant derived promoter capable of controlling expression of polypeptides in plants. Generally, promoters obtained from dicotyledonous plant species will be used when a dicotyledonous plant is selected in accordance herewith, while a monocotyledonous plant promoter will be used when a monocotyledonous plant species is selected. Constitutive promoters that may be used include, for example, the 35S cauliflower mosaic virus (CaMV) promoter (Rothstein et al., 1987, Gene 53: 153-161), the rice actin promoter (McElroy et al., 1990, Plant Cell 2:163-171; U.S. Pat. No. 6,429,357), a ubiquitin promoter, such as the corn ubiquitin promoter (U.S. Pat. Nos. 5,879,903; 5,273,894), and the parsley ubiquitin promoter (Kawalleck, P. et al., 1993, Plant Mol. Biol. 21:673-684).

Certain genetic elements capable of enhancing expression of the ABR17 protein may be used herein. These elements include the untranslated leader sequences from certain viruses, such as the AMV leader sequence (Jobling and Gehrke, 1987, Nature, 325: 622-625) and the intron associated with the maize ubiquitin promoter (U.S. Pat. No. 5,504,200). Generally the chimeric nucleic acid sequence will be prepared so that genetic elements capable of enhancing expression will be located 5′ to the nucleic acid sequence encoding the ABR17 protein.

In accordance with the present invention the chimeric nucleic acid sequences comprising a promoter capable of controlling expression in plant linked to a nucleic acid sequence encoding an ABR17 protein can be integrated into a recombinant expression vector which promotes good expression in the cell. Accordingly, the present invention includes recombinant expression vectors comprising the chimeric nucleic acid sequences of the present invention, wherein the expression vector is suitable for expression in a plant cell. The term “suitable for expression in a plant cell” means that the recombinant expression vector comprises a chimeric nucleic acid sequence of the present invention linked to genetic elements required to achieve expression in a plant cell. Genetic elements that may be included in the expression vector in this regard include a transcriptional termination region, one or more nucleic acid sequences encoding marker genes, one or more origins of replication and the like. In preferred embodiments, the expression vector further comprises genetic elements required for the integration of the vector or a portion thereof in the plant cell's nuclear genome, for example the T-DNA left and right border sequences which facilitate the integration into the plant's nuclear genome in embodiments of the invention in which plant cells are transformed using Agrobacterium.

In one embodiment, the recombinant expression vector generally comprises a transcriptional terminator which besides serving as a signal for transcription termination further may serve as a protective element capable of extending the mRNA half life (Guarneros et al., 1982, Proc. Natl. Acad. Sci. USA, 79: 238-242). The transcriptional terminator is generally from about 200 nucleotides to about 1000 nucleotides and the expression vector is prepared so that the transcriptional terminator is located 3′ of the nucleic acid sequence encoding ABR17. Termination sequences that may be used herein include, for example, the nopaline termination region (Bevan et al., 1983, Nucl. Acids. Res., 11: 369-385), the phaseolin terminator (van der Geest et al., 1994, Plant J. 6: 413-423), the arcelin terminator (Jaeger G D, et al., 2002, Nat. Biotechnol . 20:1265-8), the terminator for the octopine synthase genes of Agrobacterium tumefaciens or other similarly functioning elements. Transcriptional terminators may be obtained as described by An (An, 1987, Methods in Enzym. 153: 292).

In one embodiment, the expression vector may further comprise a marker gene. Marker genes that may be used include all genes that allow the distinction of transformed cells from non-transformed cells, including all selectable and screenable marker genes. A marker gene may be a resistance marker such as an antibiotic resistance marker against, for example, kanamycin (U.S. Pat. No. 6,174,724), ampicillin, G418, bleomycin, hygromycin or spectinomycin which allows selection of a trait by chemical means or a tolerance marker against a chemical agent, such as the normally phytotoxic sugar mannose (Negrotto et al., 2000, Plant Cell Rep. 19: 798-803). Other convenient markers that may be used herein include markers capable of conveying resistance against herbicides such as glyphosate (U.S. Pat. Nos. 4,940,935; 5,188,642), phosphinothricin (U.S. Pat. No. 5,879,903) or sulphonyl ureas (U.S. Pat. No. 5,633,437). Resistance markers, when linked in close proximity to nucleic acid sequence encoding the apolipoprotein polypeptide, may be used to maintain selection pressure on a population of plant cells or plants that have not lost the nucleic acid sequence encoding the ABR17 protein. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

Recombinant vectors suitable for the introduction of nucleic acid sequences into plants include Agrobacterium and Rhizobium based vectors, such as the Ti and Ri plasmids, including for example pBIN19 (Bevan, Nucl. Acid. Res., 1984, 22: 8711-8721), pGKB5 (Bouchez et al., 1993, C R Acad. Sci. Paris, Life Sciences, 316:1188-1193), the pCGN series of binary vectors (McBride and Summerfelt, 1990, Plant Mol. Biol., 14:269-276) and other binary vectors (e.g. U.S. Pat. No. 4,940,838).

The recombinant expression vectors of the present invention may be prepared in accordance with methodologies well known to those skilled in the art of molecular biology. Such preparation will typically involve the bacterial species Escherichia coli as an intermediary cloning host. The preparation of the E. coli vectors as well as the plant transformation vectors may be accomplished using commonly known techniques such as restriction digestion, ligation, gelectrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13mp series of vectors, pBluescript etc. Typically, these cloning vectors contain a marker allowing selection of transformed cells. Nucleic acid sequences may be introduced in these vectors, and the vectors may be introduced in E. coli grown in an appropriate medium. Recombinant expression vectors may readily be recovered from cells upon harvesting and lysing of the cells. Further, general guidance with respect to the preparation of recombinant vectors may be found in, for example: Sambrook et al., Molecular Cloning, a Laboratory Manual, Cold Spring Harbor Laboratory Press, 1989, Vol. 3.

The expression vector comprising a nucleic acid sequence encoding ABR 17 is then transfected into the appropriate plant cell. Examples of how this can be accomplished are provided in the Examples herein. As non-limiting examples, the expression vector may be transfected into Arabidopsis, Brassica or cells from other plants, including monocots and dicots, including wheat, barley, rice, maize, potato and cotton.

Methodologies to introduce plant recombinant expression vectors into a plant cell, also referred to herein as “transformation”, are well known to the art and typically vary depending on the plant cell that is selected. General techniques to introduce recombinant expression vectors in cells include, electroporation; chemically mediated techniques, for example CaCl₂ mediated nucleic acid uptake; particle bombardment (biolistics); the use of naturally infective nucleic acid sequences, for example virally derived nucleic acid sequences, or Agrobacterium or Rhizobium derived sequences, polyethylene glycol (PEG) mediated nucleic acid uptake, microinjection and the use of silicone carbide whiskers.

In preferred embodiments, a transformation methodology is selected which will allow the integration of the chimeric nucleic acid sequence in the plant cell's genome, and preferably the plant cell's nuclear genome. The use of such a methodology is preferred as it will result in the transfer of the chimeric nucleic acid sequence to progeny plants upon sexual reproduction. Transformation methods that may be used in this regard include biolistics and Agrobacterium mediated methods.

Transformation methodologies for dicotyledenous plant species are well known. Generally, Agrobacterium mediated transformation is used because of its high efficiency, as well as the general susceptibility by many, if not all, dicotyledenous plant species. Agrobacterium transformation generally involves the transfer of a binary vector, such as one of the hereinbefore mentioned binary vectors, comprising the chimeric nucleic acid sequence of the present invention from E. coli to a suitable Agrobacterium strain (e.g. EHA101 and LBA4404) by, for example, tri-parental mating with an E. coli strain carrying the recombinant binary vector and an E. coli strain carrying a helper plasmid capable of mobilizing the binary vector to the target Agrobacterium strain, or by DNA transformation of the Agrobacterium strain (Hofgen et al., Nucl. Acids. Res., 1988, 16:9877). Other techniques that may be used to transform dicotyledenous plant cells include biolistics (Sanford, 1988, Trends in Biotechn. 6:299-302); electroporation (Fromm et al., 1985, Proc. Natl. Acad. Sci. USA., 82:5824-5828); PEG mediated DNA uptake (Potrykus et al., 1985, Mol. Gen. Genetics, 199:169-177); microinjection (Reich et al., Bio/Techn., 1986, 4:1001-1004); and silicone carbide whiskers (Kaeppler et al., 1990, Plant Cell Rep., 9:415-418) or in planta transformation using, for example, a flower dipping methodology (Clough and Bent, 1998, Plant J., 16:735-743).

Monocotyledonous plant species may be transformed using a variety of methodologies including particle bombardment (Christou et al., 1991, Biotechn. 9:957-962; Weeks et al., Plant Physiol., 1993, 102:1077-1084; Gordon-Kamm et al., Plant Cell, 1990, 2:5603-618); PEG mediated DNA uptake (European Patents 0292 435; 0392 225) or Agrobacterium mediated transformation (Goto-Fumiyuki et al., 1999, Nature-Biotech. 17:282-286).

The exact plant transformation methodology may vary somewhat depending on the plant species and the plant cell type (e.g. seedling derived cell types such as hypocotyls and cotyledons or embryonic tissue) that is selected as the cell target for transformation. As mentioned above, in a particularly preferred embodiment, Brassica napus is used. A methodology to obtain safflower transformants is described in Baker and Dyer (Plant Cell Rep., 1996, 16:106-110). Additional plant species specific transformation protocols may be found in: Biotechnology in Agriculture and Forestry 46: Transgenic Crops I (Y.P.S. Bajaj ed.), Springer-Verlag, New York (1999), and Biotechnology in Agriculture and Forestry 47: Transgenic Crops II (Y.P.S. Bajaj ed.), Springer-Verlag, New York (2001).

Following transformation, the plant cells are grown and upon the emergence of differentiating tissue, such as shoots and roots, mature plants are regenerated. Typically a plurality of plants is regenerated. Methodologies to regenerate plants are generally plant species and cell type dependent and will be known to those skilled in the art. Further guidance with respect to plant tissue culture may be found in, for example: Plant Cell and Tissue Culture, 1994, Vasil and Thorpe Eds., Kluwer Academic Publishers; and in: Plant Cell Culture Protocols (Methods in Molecular Biology 111), 1999, Hall Eds, Humana Press.

### EXAMPLES

The following examples are intended only to illustrate and describe the invention rather than limit the claims that follow.

### Example 1

### Arabidopsis thaliana

Plant Expression Vectors

A pea ABR17 cDNA clone was amplified using as a forward primer having SEQ ID NO: 3: 5′-GTGGTCGAAGCTTATGGGTGTCTTTGTTTTTGATGATGAATAC-3′. This primer, beginning with the ATG (underlined) corresponds to nucleotides 20-49 inclusive, of SEQ ID NO: 1. The sequence of the reverse primer follows, and is SEQ ID NO: 4: 5′-TATATAGCTCGAGTTAGTAACCAGGATTTGCCAAAACGTAACC-3′. This primer, beginning with the TTA (underlined) corresponds to the reverse complement of nucleotides 493-464 inclusive, of SEQ ID NO: 1.

The restriction enzyme sites Hind III and Xho I are highlighted on the forward and reverse primers, respectively. The amplified cDNA fragment was ligated between the CaMV35S promoter and the rbcS3′ terminator in the binary vector pKYLX71 (Schald et al., 1987) and the resulting gene construct was sequenced to ensure ligation of the cDNA in the correct orientation and the absence of any mutations/rearrangement.

The gene construct was subsequently introduced into the disarmed Agrobacterium tumefaciens strain GV3101 through a triparental mating technique using E. coli strain HB101 carrying the helper plasmid pRK2013. Transconjugant A. tumefaciens was selected on solid medium containing rifampicin, gentamicin and tetracycline. Plasmid DNA was extracted from A. tumefaciens and analyzed by restriction digestion to ensure that no rearrangements had taken place during the introduction into this bacterium.

Transformation of Arabidopsis thaliana

A. thaliana ecotype WS was transformed using the floral dip method (Clough and Bent, 1998). Briefly, plants were grown in growth chambers at 25° C. until flowering with a 16 h light/8 h dark photoperiod. Primary racemes were clipped to encourage proliferation of secondary bolts. A. tumefaciens cells at mid-log stage of growth were centrifuged and resuspended to a density of 0.8 (Abs₆₀₀) in 5% sucrose solution containing 0.05% silwet L-77. Above-ground parts of A. thaliana plants were dipped in this Agrobacterium solution with gentle agitation. Dipped plants were covered with plastic wrap (for 16-24 h) to maintain high humidity and were returned to growth chambers. Harvested, dry seeds (T0) were surface sterilized and placed on ½ strength MS plates containing 50 mg/L kanamycin. The seeds were cold treated at 4° C. for at least 2 d, and grown at 25° C. with 16 h light/8 h dark, 100 μE light for 7-10 d. Plants that survived on the kanamycin plates were transferred to soil and maintained in growth cabinets. A number of independent transgenic lines were tested for their abilities to germinate on MS plates containing 75 or 150 mM NaCl and, those with enhanced germination were selected for the production of homozygous plants.

The number of copies of the ABR17 cDNA in these lines was estimated based on their segregation on kanamycin plates. Essentially, seeds from the transgenic plants (T1 seeds) were screened on kanamycin plates to determine segregation ratios and those with a monogenic (3:1) segregation ratio and exhibiting enhanced germination in the presence of NaCl were used for homozygous T2 seed production. Bulked homozygous seeds from T2 plants were used in all subsequent experiments.

Three transgenic lines (6.9, 14.9 and 25.20) with single insertions of the ABR17 cDNA that showed best germination in preliminary experiments under saline conditions were selected for the production of homozygous lines and further characterization.

RT-PCR Analysis

Total RNA, isolated from wild type and transgenic seedlings, was reverse transcribed and subsequently amplified to detect ABR17 transcripts. RNA was isolated using the QIAGEN RNeasy Plant Mini Kit (Qiagen, Mississauga, ON, Canada) from pooled 2 week-old seedling tissue. The isolated RNA was treated with RNase-free DNase (Qiagen) to ensure the complete removal of DNA prior to RT-PCR. Reverse transcription and first strand cDNA synthesis of total RNA (50 ng) was performed using the iScript cDNA synthesis kit (Bio-Rad, Hercules, Calif., USA). PCR reactions were carried out using the newly synthesized cDNA (2 μL) as the template and as the forward primer 5′-GGTGATCAAGGAAGCACAAGG-3′ [SEQ ID NO: 5] and as the reverse primer 5′-TTTGGCCTTTGTTTCATCACG-3′ [SEQ ID NO: 6] specific to the pea ABR17 coding sequence using a PCR Master Mix (Promega, Md., USA). The ABR17 transcript was amplified using the following thermocycling parameters: 94° C., 2 min; 35 cycles at 94° C., 1 min; 62° C., 1 min; 72° C., 1 min; and a final extension of 72° C., 10 min. Plant 18s rRNA primers, with the forward being 5′-CCAGGTCCAGACATAGTAAG-3′ [SEQ ID NO: 7] and reverse being 5′-GTACAAAGGGCAGGGACGTA-3′ [SEQ ID NO: 8] (Duval et al., 2002), were used as an internal controls. PCR products were separated by electrophoresis on a 1.2% agarose gel and visualized under UV light after staining with ethidium bromide.

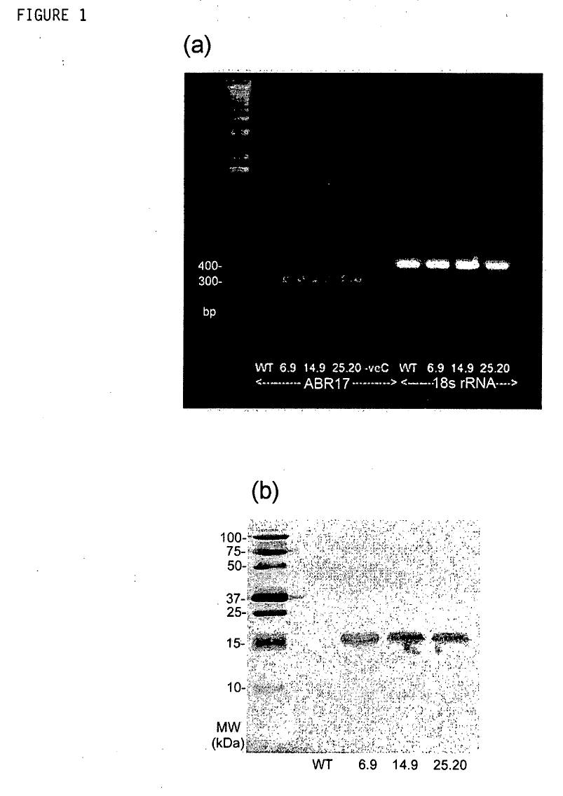

The presence of an ABR17-specific transcript was confirmed by this RT-PCR analysis, which produced the expected 319 bp amplification product that was absent in the wild type as well as the negative control (FIG. 1(a)). The quality of mRNA, cDNA and the amplification reactions was confirmed by the successful amplification of the expected 18s rRNA product in all samples (FIG. 1(a)).

Western Blot Analysis

Two week-old pooled Arabidopsis seedlings were crushed in liquid N₂ and 500 μL extraction buffer (0.5 M Tris-HCl, pH 6.8; containing 10% glycerol; 10% SDS and 60 mM DTT) was added to 200 mg ground tissue. The tubes were vortexed and a small amount of protamine sulfate on the tip of a spatula was added and incubated at RT for 15 min prior to centrifugation at 12 500 g for 15 min. Ice-chilled acetone containing 0.07% DTT (5 equal volumes) was added to the supernatants and they were centrifuged once more as described above. The pellets were dried for 15 min under vacuum and resuspended in 100 μL of 50 mM Tris pH 6.8 containing 0.5% SDS. The concentration of protein in the resuspended samples was determined using modified Bradford assay and the samples stored at −20° C. until subjected to Western blot analysis.

Protein (30 μg) was applied to polyacrylamide gels (15%) and subjected to electrophoresis according to Laemmli (1970) using a Mini PROTEAN 3 vertical slab system (Bio-Rad) at constant 160 V until the dye front reached the bottom of the gel. After electrophoresis the gel was equilibrated in transfer buffer (48 mM Tris, pH 9.2 containing 39 mM glycine, 20% methanol and 1.3 mM SDS) for 15 min and transferred to polyvinylidene fluoride (PVDF) membrane for 25 min at 15 V using Trans Blot SD, semi-dry transfer apparatus (Bio-Rad). Following transfer, the membrane was blocked with TBS (10 mM Tris-HCl, pH 7.5; 150 mM NaCl) containing 5% non-fat skim milk powder for 2 h and rinsed with TTBS (TBS containing 0.05% Tween 20) for 10 min. The primary antibody solution (rabbit anti-PR 10.4 in TTBS; 1:20,000) was added to the membrane and, after 1 h incubation, the membrane was washed 3 times for 5 min each with TTBS. The membrane was then incubated with secondary antibody solution [goat anti-rabbit immunoglobulin G conjugated with horseradish peroxidase (Abcam, Mass., USA) in TTBS; 1:10,000] for 1 h and membrane after which the membrane was washed 3 times for 5 min each with TTBS followed by a 5 min wash with TBS. Bands on the blot were visualized by staining the membrane with TMB peroxidase substrate kit (Vector laboratories Inc., California, USA) according to manufacturer's instructions.

The presence of the ABR17 protein in all the three transgenic lines was confirmed by Western blot analysis with PR 10.4-specific polyclonal antibodies. Protein extracts prepared from the transgenic plants revealed the presence of a strongly reacting band with the molecular weight corresponding to that of ABR17, whereas extracts from the wild type plants did not show the presence of this band (FIG. 1(b)).

Plant Growth and Germination Experiments

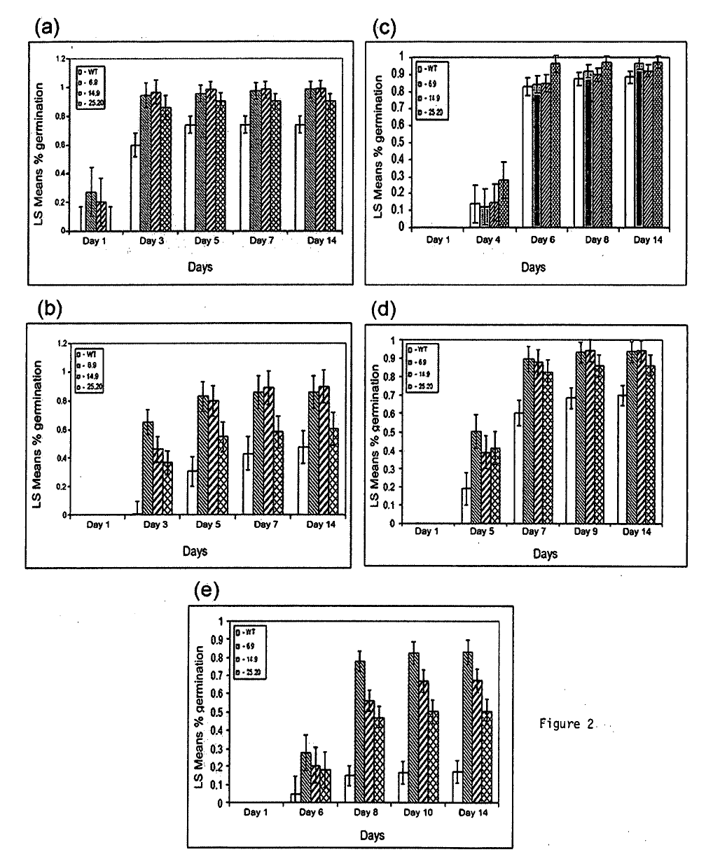

Wild type and transgenic lines (6.9, 14.9, 25.20) of A. thaliana, ecotype WS seeds were grown on ½ strength MS medium containing 1.5% sucrose, 0.8% agar, pH 5.7 and various salt concentrations (0, 75 and 150 mM NaCl) in 25×100 mm Petri dishes. Seeds were surface sterilized by immersing in 70% ethanol for 1 min followed by rinsing twice with sterile deionized water. The seeds were then placed in a solution of 20% bleach for 15 min with occasional mixing, after which they were rinsed four times (5 min each) with sterile deionized water. At least 5 plates per treatment and 14 seeds per plate were used in these experiments and plates were incubated under continuous light either at 22° C. or 10° C. Germination of seeds was monitored every day and the numbers of germinated seeds were recorded daily for two weeks. Those seeds where the radicles had emerged were considered to have germinated. Each experiment was repeated at least three times and the results are shown in FIG. 2.

At room temperature, in the absence of NaCl, 100% germination in the wild type as well as the three transgenic lines, was observed (data not shown). At the lower temperature (10° C.), in the absence of NaCl, while the germination in all the lines was not 100%, there were no significant differences in the numbers of germinated seeds on any day (FIG. 2(c)).

In the presence of 75 and 150 mM NaCl at both temperatures tested, the transgenic seedlings germinated earlier. For instance, the transgenic lines 6.9 and 14.9 initiated germination by day 1 in presence of 75 mM NaCl whereas no wild type or 25.20 transgenic seeds had germinated at this point (FIG. 2(a)). Similarly, in the presence of 150 mM NaCl, all three transgenic lines had initiated germination by day 3 whereas no significant germination in the wild type had occurred at this time (FIG. 2(b)). In the presence of 75 mM NaCl the maximum germination observed at room temperature at the conclusion of the experiment (on day 14) in the wild type was ˜74%, compared to that of 99%, 99%, 90% for the transgenic lines 6.9, 14.9 and 25.20, respectively (FIG. 2(a)). In the case of 150 mM NaCl, the maximum germination in the wild type was 47% compared to 85%, 89% and 60% in the transgenic lines 6.9, 14.9 and 25.20, respectively.

When the germination of the seeds was assessed at 10° C., in the presence of either 75 or 150 mM NaCl, the differences in germination rates of the transgenic seeds were obvious with the transgenic lines clearly performing better than the wild type (FIGS. 2(d) & (e)). At 10° C. on day 5, in the presence of 75 mM NaCl, all three transgenic lines had significantly higher germination rate compared to wild type, with the transgenic line 6.9 appearing to be the best among the transgenics (FIG. 2(d)). The trend remained the same throughout the entire period of the experiment (14 days) for 75 mM NaCl treatments. In the presence of 75 mM NaCl and 10° C., at the end of the 14-day experiment, 70% of the wild type seeds had germinated, whereas the germination rates were 94%, 94% and 86% in the three transgenic lines 6.9, 14.9 and 25.20, respectively (FIG. 2(d)). In the presence of 150 mM NaCl, the difference between the wild type and the three transgenics was apparent on day 6, and was very clear on day 8 (FIG. 2(e)). At this salt concentration, only 17% of the wild type seeds had germinated by day 14, whereas the germination rate was 83%, 67% and 50% in the three transgenic lines 6.9, 14.9 and 25.20, respectively. Once again, the transgenic line 6.9 appeared to be better than the other two lines (FIG. 2(e)). All of the observed differences between the germination rates of the transgenic and wild type seeds were statistically significant (P<0.05).

Appearance of Seedlings Germinated and Grown in the Presence of NaCl

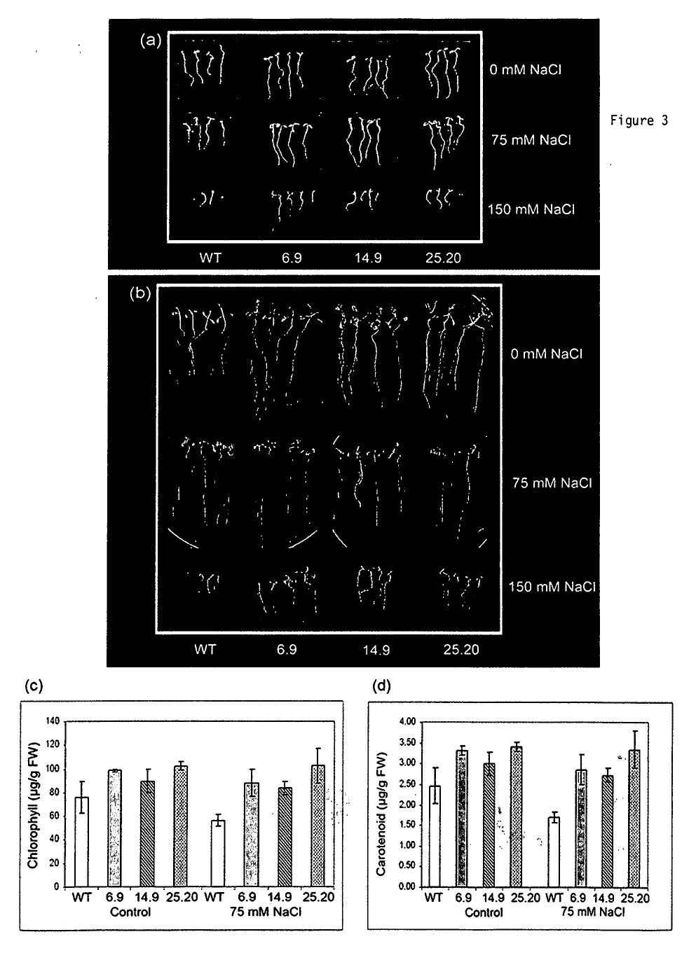

The appearance of the wild type and transgenic seedlings grown on MS plates containing 0, 75 or 150 mM NaCl at room temperature, after one and two weeks, is shown in FIGS. 3(a) & (b), respectively. There are no obvious differences between the wild type and 3 transgenic lines in the absence of salt after one week of growth (FIG. 3(a)). However, after 2 weeks, in the absence of salt, it appears as though both roots and shoots of the transgenic seedlings are better developed compared to the wild type (FIG. 3(b)). As previously described for the germination experiments, the differences between the wild type and transgenic seedlings were more apparent in the presence of NaCl (FIG. 3), with the deleterious effects on root and shoot development being more pronounced at the higher (150 mM) NaCl concentration. It is clear from the appearance of the seedlings (FIG. 3) that the deleterious effects of 150 mM NaCl at both 1 and 2 weeks in the transgenic lines are considerably less compared to those on the wild type seedlings. These observations were confirmed by evaluating the effect of the salinity treatments on the chlorophyll and carotenoids levels, which were measured in 2 week-old control as well as 75 mM NaCl treated tissues.

Measurement of Chlorophyll, Carotenoid and Ion-Leakage

The chlorophyll and carotenoid content as well as ion-leakage were used to assess plant damage due to the stresses. Total chlorophyll and carotenoid was extracted from pooled tissue of 2 week-old plants grown on MS plates, using a procedure modified from Kirk and Allen (1965). Pooled leaf tissue (0.05 g) was homogenized in 5 mL of ice-cold 80% acetone and incubated at room temperature for 10 min in the dark. Samples were then centrifuged at 2 500 g for 15 min. The supernatant was decanted and absorbance measured at 663, 645 and 480 nm. Total chlorophyll (a+b) was estimated using a nomogram (Kirk 1968) and the carotenoid levels were calculated using the formula (Kirk and Allen, 1965):
ΔACAR₄₈₀=ΔA₄₈₀+0.114 ΔA₆₆₃−0.638 ΔA₆₄₅ where, A is absorbance and CAR is carotenoid content.

Membrane damage was assessed by measuring ion-leakage from cold-stressed leaves (Vettakkorumakankav et al., 1999). Leaves from control and stressed plants were incubated in 20 mL of distilled water and agitated at room temperature for an hour following which the conductivity of the solution (initial value) was determined using a conductivity meter (HI 8733, Hanna Instrument, Rhode Island, USA). The solutions were incubated at 4° C. overnight and subsequently autoclaved to release total ions and the final conductivity values were determined. Ion leakage is expressed as a percentage of the initial to final values.

The chlorophyll levels were significantly (P<0.05) reduced in the wild type seedlings whereas the levels in the transgenics grown in 75 mM NaCl were not significantly different from the untreated samples (FIG. 3(c)). The effect of 75 mM NaCl was more pronounced on the carotenoid levels of the wild type seedlings, with the salt treatment reducing the carotenoid levels significantly (P<0.05) compared to the transgenics (FIG. 3(d)). It is also apparent that the carotenoid levels in the untreated transgenic seedlings were higher compared to that of the untreated wild type seedlings (FIG. 3(d)). These elevated levels of carotenoids may be contributing to the better tolerance to NaCl exhibited by the transgenic lines.

Appearance of Seedlings Germinated and Grown at 10° C.

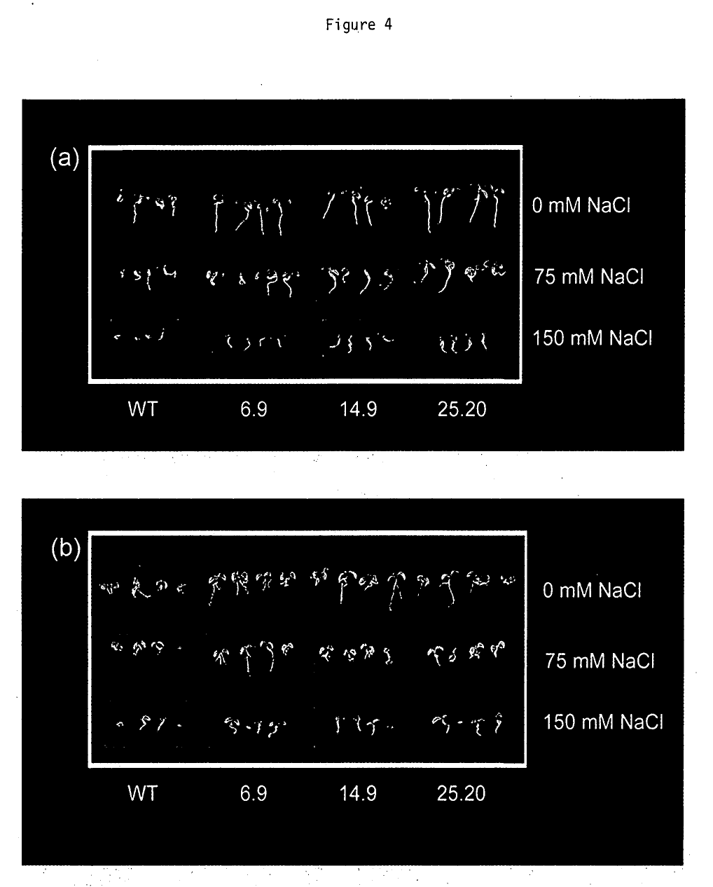

The appearance of seedlings after two and three weeks at 10° C. in the presence or absence of NaCl is shown in FIG. 4. It is evident that at this lower temperature, in the presence or absence of NaCl, the transgenic seedlings appear to be healthier than the wild type after 2 weeks (FIG. 4(a)) and 3 weeks (FIG. 4(b)) of growth, suggesting that they are able to better tolerate the lower temperature stress. Therefore, the appearance of the seedlings also support the data obtained from the experiments that assessed the abilities of the transgenic lines to germinate under these stress conditions.

Imposition of Stresses

Wild-type and transgenic Arabidopsis lines (6.9, 14.9 and 25.20) were evaluated for tolerance to cold and high temperature stresses as described by Kim et al. (2004). For the imposition of the cold stress open MS plates containing the plants were placed at −5° C. (freezing temperature) for 4 h after which the plates were returned to room temperature and photographed after 24 h of recovery. In the case of heat stress, 2 week-old plants in closed MS plates were placed in an incubator set at 48° C. for 2 h after which they were returned to room temperature and photographed after 1 and 3 days of recovery.

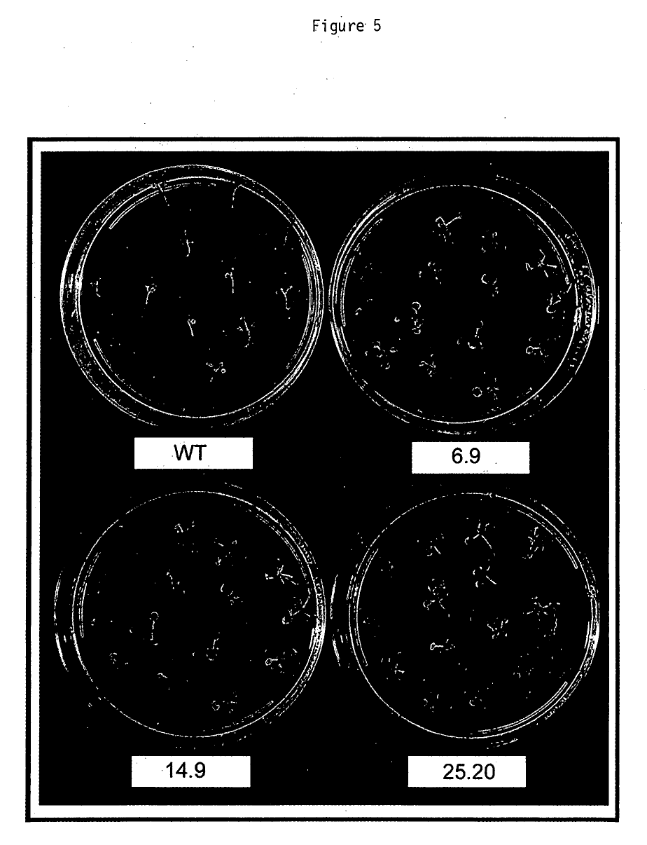

The appearance of plants grown on MS plates 24 h after a 4 h treatment at −5° C. is shown in FIG. 5. It is apparent that almost all the wild type plants collapsed, whereas the transgenic lines appeared healthier indicating that these lines were more tolerant to the imposed stress (FIG. 5). In order to confirm that this was indeed the case, the percent ion leakage in the wild type and transgenic seedlings after 4 h of stress and 4 h of incubation at 4° C., as well as after the 24 h recovery at room temperature (Table 1), was determined.

TABLE 1
Effects of cold stress on ion leakage, chlorophyll and carotenoid content.
Percentage Ion leakage
Cold stress −5° C. for 4 h and 4 h incubation at 4° C.

| | |
|---|---|
| WT | 61.69 ± 3.32 |
| 6.9 | 37.17 ± 3.66 |
| 14.9 | 47.22 ± 1.49 |
| 25.20 | 44.84 ± 1.17 |

Percentage Ion leakage
1 day recovery after cold stress −5° C. for 4 h

| | |
|---|---|
| WT | 51.36 ± 4.17 |
| 6.9 | 38.26 ± 4.41 |
| 14.9 | 44.57 ± 6.94 |
| 25.20 | 38.31 ± 7.40 |

Chlorophyll (μg/g FW)
1 day recovery after cold stress −5° C. for 4 h

| | |
|---|---|
| WT | 72.45 ± 1.02 |
| 6.9 | 98.47 ± 18.93 |
| 14.9 | 81.83 ± 4.29 |
| 25.20 | 74.41 ± 15.64 |

Carotenoid (μg/g FW)
1 day recovery after cold stress −5° C. for 4 h

| | |
|---|---|
| WT | 2.50 ± 0.05 |
| 6.9 | 2.95 ± 0.56 |
| 14.9 | 2.68 ± 0.16 |
| 25.20 | 2.37 ± 0.56 |

Table 1 indicates that the percent ion leakage, which is indicative of membrane damage, after the stress and 4° C. incubation, was significantly less in all three transgenic lines compared to the wild type, even though it appears as if line 6.9 suffered the least amount of membrane damage when compared to the other two transgenic lines. These observations are also supported by the ion leakage observed after the 24 h recovery period where, once again, it was observed that line 6.9 had the least amount of membrane damage. Even though the appearance, as well as the ion leakage data, indicated that the transgenic plants were significantly more tolerant to the cold stress, there were no significant differences in the chlorophyll or carotenoids content when measured after the stress as well as after the recovery period (Table 1).

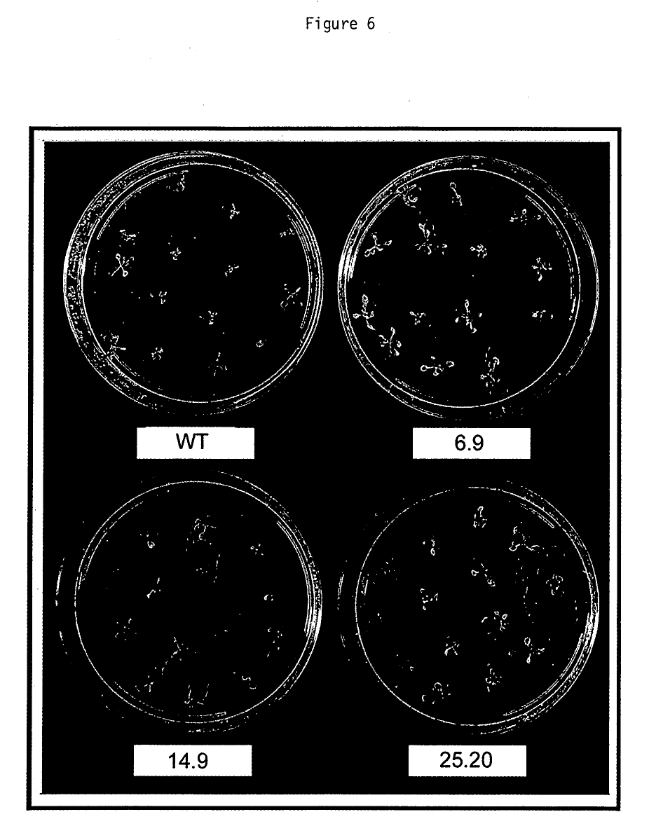

The enhanced germination of the transgenic lines in the presence of NaCl and the increased tolerance of these lines to cold temperature stress prompted testing of whether the ABR17 transgenic plants would exhibit enhanced tolerance to heat stress. The appearance of these plants after the one-day recovery period is shown in FIG. 6. It is evident that all three transgenic lines appeared healthier compared to the wild type after one day of recovery suggesting that ABR17 expression could confer multiple stress tolerance.

Two-Dimensional Electrophoresis

In order to probe the physiological basis for the observed enhancement of stress tolerance in transgenic A. thaliana seedlings expressing the pea ABR17 gene we performed two-dimensional electrophoresis to compare proteome-level changes brought about by the transgene expression. Protein extracts for two-dimensional electrophoresis were prepared according to the method described by Subramanian et al. (2005) with some modifications. Wild-type and transgenic seedlings (two week-old) from MS plates were homogenized to a fine powder in liquid nitrogen. Homogenized tissue (0.3 g) was further homogenized in acetone containing 10% (w/v) TCA and 0.07% DTT, transferred to eppendorf tubes and the volume was adjusted to 1.5 mL with acetone containing 10% (w/v) TCA and 0.07% DTT. Samples were incubated at −17° C. for 1 h, centrifuged at 13 000 g for 15 min and the supernatants discarded. The pellets were washed by resuspending them in ice-cold acetone containing 0.07% DTT and centrifuged as described above. This was step was repeated four additional times, the pellets were dried at room temperature in a speed vac for 30 min and resuspended in 400 μL Rehydration/Sample buffer (Bio-Rad, Ontario, Canada) containing 8 M urea, 2% w/v CHAPS, 40 mM DTT, 0.2% Bio-lyte 3-10 and 3 μL of 200 mM tributylphosphine (TBP). The samples were mixed vigorously, incubated overnight at 4° C. and centrifuged at 4° C. for 15 min at 13 000 g. The supernatants were placed in fresh tubes and protein concentrations determined using a modified Bradford assay (Bio-Rad) using BSA as standard.

Two-dimensional electrophoresis of protein extracts was performed as previously described (Subramanian et al., 2005). Briefly, immobilized pH gradient (IPG) strips (17 cm, Bio-Rad) were passively rehydrated overnight with 300 μg of protein in 300 μL of Rehydration buffer (8 M urea, 2% CHAPS, 40 mM DTT, 0.2% Bio-Lyte and 2 mM TBP). Isoelectric focusing (IEF) was performed using a Bio-Rad PROTEAN IEF unit to provide an optimum, maximum field strength of 600 V/cm and a 50 μA limit/IPG strip at 10000 V for 60000 VH. Prior to second dimension separation, proteins in the rehydrated strips were reduced by incubating them twice in a solution (5 mL/strip) containing 6 M urea, 2% sodium dodecyl sulfate (SDS), 0.375 M Tris-HCl, pH 8.8, 20% glycerol, and 130 mM DTT for 10 min each. The strips were then incubated in the above solution containing 135 mM iodoacetamide instead of DTT twice for 10 min each in order to alkylate the reduced proteins. SDS-PAGE separation of proteins was performed on 13% polyacrylamide gels (20×20 cm, 1 mm thickness) using a PROTEAN II XI system (Bio-Rad) at constant voltage (90 V) until the dye front reached the bottom of the gel. Protein spots were visualised using a Colloidal Blue Staining Kit (Invitrogen, Calif., USA) according to the instructions provided. Two-dimensional electrophoresis was performed at least three times with the extracted protein samples.

Image Analysis

Images of the two-dimensional gels were acquired using a GS-800 calibrated densitometer (Bio-rad) and analyzed using the PDQuest software (Bio-rad). Three gels each of wild type and transgenic (6.9) samples were used to generate the match-sets and individual spots were matched using automated detection and matching feature of the software followed by manual refinements in order to eliminate artifacts and include spots that were missed by the automated detection process. In order to identify the protein spots whose levels were significantly different between the transgenic and wild type seedlings, the match sets from 3 replicate gels of wild type and 6.9 samples were analyzed using Student's t-test feature of PDQuest software as described by the manufacturer. Those spots which were reproducibly altered in all three replicates and exhibited significant (P<0.01) difference were excised from the gels using sterile scalpels and subjected to ESI-Q-TOF-MS/MS analysis.

ESI-Q-TOF MS/MS Analysis

Tandem MS was performed at the Institute for Biomolecular Design, University of Alberta, on protein extracted from isolated gel spots as previously described (Subramanian et al., 2005). Briefly, gel pieces were de-stained, reduced with 10 mM DTT, alkylated with 55 mM iodoacetamide and digested with 6 ng/μl trypsin (Promega Sequencing Grade Modified) in 50 mM ammonium bicarbonate (25 μl), for 5 h at 37° C. in a fully automated fashion on a Mass Prep Station (Micromass, Manchester, UK). The tryptic peptides were subjected to LC/MS/MS analysis on a Micromass Q-ToF-2 mass spectrometer (Micromass) coupled with a Waters CapLC capillary HPLC (Waters Corp., USA). Peptides were separated on a PicoFrit capillary reversed-phase column (5μ BioBasic C18, 300 Angstrom pore size, 75μ ID×10 cm, 15μ tip (New Objectives, MA, USA), using a linear water/acetonitrile gradient (0.2% Formic acid), after desalting on a 300μ×5 mm PepMap C18 column (LC Packings, California, USA). Eluent was introduced directly to the mass spectrometer by electrospray ionization at the tip of the capillary column and data dependent MS/MS acquisition was performed for peptides with a charge state of 2 or 3. Proteins were identified from the MS/MS data by searching the NCBI non-redundant database with Mascot Daemon (Matrix Science, UK) including carbamidomethylation of cysteine, possible oxidation of methionine and one missed cleavage per peptide as search parameters.

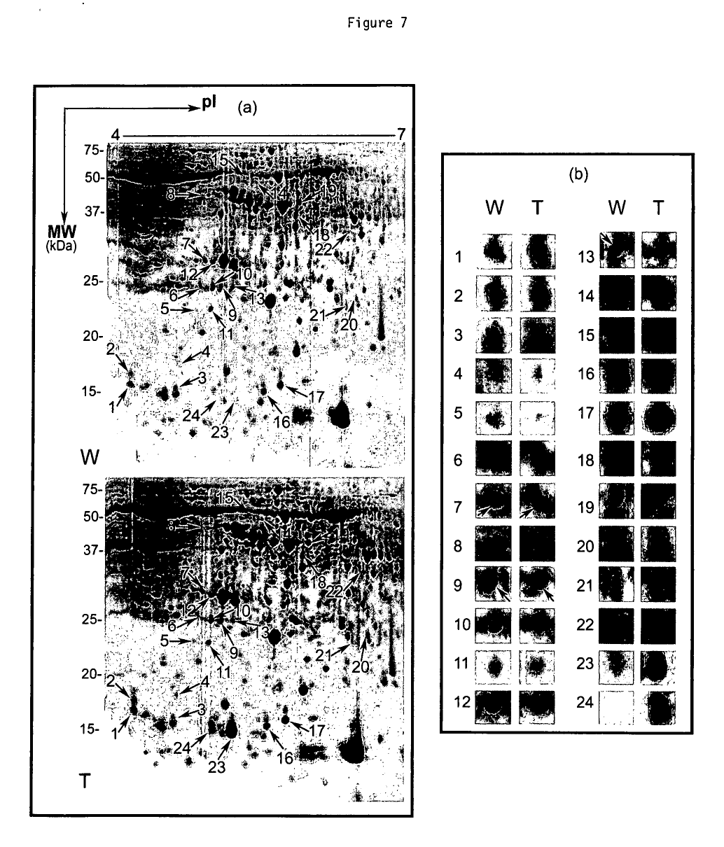

Representative images of two-dimensional gels obtained from wild type and ransgenic protein extracts are shown in FIG. 7(a). The intensities of 24 protein spots FIG. 7(b)) were observed to be significantly (P<0.01) and reproducibly altered in transgenic ine (6.9) compared to the wild type. Out of these 24 protein spots, the intensities of 7 were reater, 15 were lower and two spots were unique in the transgenic line. The identities of hese 24 spots were established using tandem MS and are presented in Table 2.

TABLE 2

Details of proteins identified by ESI-Q-TOF MS/MS

MS/MS ESI-Q-ToF

| | | |
|---|---|---|
| Spot | ^{a}PM | Fold |

| | | | | | | | |
|---|---|---|---|---|---|---|---|
| No. | Protein identity | % | ^{b}score | Sequence | ^{c}Access. No. | Mr/pI | change |

| | | | | | | | |
|---|---|---|---|---|---|---|---|
| 1 | DRT112 | 14% | 88 | NNAGYPHNVVFDEDEIPSGVDVAK | gi\|166696 | 17089/5.06 | 3.49 ± 0.57 |
| | [Arabidopsis | | (>48) | | | | ↑ |
| | thaliana] | | | | | | |
| 2 | Putative | 23% | 153 | AAEDPAPASSSSK | gi\|24030202 | 14756/9.92 | 18.17 ± 10.23 |
| | photosystem I | | (>49) | RESYWFK | | | ↑ |
| | subunit PSI-E | | | NVGSVVAVDQDPK | | | |
| | protein | | | | | | |
| | [Arabidopsis | | | | | | |
| | thaliana] | | | | | | |
| 3 | GDCH | 15% | 147 | FCEEEDAAH | gi\|15226973 | 18050/5.24 | 0.14 ± 0.13 |
| | [Arabidopsis | | (>49) | VKPSSPAELESLMGPK | | | ↓ |
| | thaliana] | | | | | | |
| 4 | Unknown protein | 7% | 85 | VEVTEAEVELGFK | gi\|18422918 | 17603/4.80 | 0.68 ± 0.07 |
| | [Arabidopsis | | (>42) | | | | ↓ |
| | thaliana] | | | | | | |
| 5 | Bas1 protein | 27% | 140 | YVILFFYPLDFTFVCPTEITAFSDR | gi\|861010 | 23398/5.48 | 0.47 ± 0.02 |
| | [Hordeum vulgare] | | (>48) | SGGLGDLKYPLVSDVTK | | | ↓ |
| | | | | EGVIQHSTINNLGIGR | | | |
| 6 | LHB1B2; | 59% | 483 | GPSGSPWYGSDR | gi\|18403546 | 28093/5.28 | 0.42 ± 0.11 |
| | chlorophyll | | (>49) | YLGPFSGEPPSYLTGEFPGDYGWDT | | | ↓ |
| | binding | | | AGLSADPETFAR | | | |
| | [Arabidopsis | | | WAMLGALGCVFPELLAR | | | |
| | thaliana] | | | FGEAVWFK | | | |
| | | | | LAMFSMFGFFVQAIVTGK | | | |
| | | | | GPLENLADHLADPVNNNAWAFATN | | | |
| | | | | FVPGK | | | |
| | | | | YLGPFSGEPPSYLTGEFPGDYGWDT | | | |
| | | | | AGLSADPETFAR | | | |
| | LHB1B1; | 60% | 445 | ASKPTGPSGSPWYGSDR | gi\|18403549 | 28209/5.15 | |
| | chlorophyll | | (>49) | YLGPFSGEPPSYLTGEFPGDYGWDT | | | |
| | binding | | | AGLSADPETFAR | | | |
| | [Arabidopsis | | | WAMLGALGCVFPELLAR | | | |
| | thaliana] | | | FGEAVWFK | | | |
| | | | | VAGDGPLGEAEDLLYPGGSFDPLGL | | | |
| | | | | ATDPEAFAELK | | | |
| | | | | LAMFSMFGFFVQAIVTGK | | | |
| | | | | GPLENLADHLADPVNNNAWAFATN | | | |
| | | | | FVPGK | | | |
| 7 | Cp29 | 27% | 499 | VNAGPPPPK | gi\|681904 | 34602/5.23 | 0.86 ± 0.05 |
| | [Arabidopsis | | (>49) | SSYGSGSGSGSGSGSGNR | | | ↓ |
| | thaliana] | | | LYVGNLSWGVDDMALENLFNEQG | | | |
| | | | | K | | | |
| | | | | GFGFVTLSSSQEVQK | | | |
| | | | | AINSLNGADLDGR | | | |
| | | | | VSEAEARPPR | | | |
| | Inorganic | 22% | 326 | VQEEGPAESLDYR | gi\|15242465 | 33644/5.71 | |
| | diphosphatase/ | | (>49) | VFFLDGSGK | | | |
| | magnesium ion | | | VSPWHDIPLTLGDGVFNFIVEIPK | | | |
| | binding/ | | | IVAISLDDPK | | | |
| | pyrophosphatase | | | HFPGTLTAIR | | | |
| | [Arabidopsis | | | IPDGKPANR | | | |
| | thaliana] | | | | | | |
| 8 | Putative aspartyl | 9% | 177 | GDLASESILLGDTK | gi\|12324588 | 47745/6.06 | 0.47 ± 0.10 |
| | protease | | (>49) | SSVSLVSQTLK | | | ↓ |
| | [Arabidopsis | | | VIYDITQER | | | |
| | thaliana] | | | LGIVGENCR | | | |
| | ribosomal protein | 8% | 151 | VSDIATVLQPGDTLK | gi\|18060 | 45044/5.41 | |
| | S1 [Spinacia | | (>49) | AEEMAQTFR | | | |
| | oleracea] | | | IAQAFAMAR | | | |
| 9 | Ribose-5- | 53% | 727 | AVEAIKPGMVLGLGTGSTAAFAVD | gi\|15229349 | 29401/5.72 | 1.46 ± 0.09 |
| | phosphate | | (>49) | QIGK | | | ↑ |
| | isomerase | | | LLSSGELYDIVGIPTSK | | | |
| | [Arabidopsis | | | SLGIPLVGLDTHPR | | | |
| | thaliana] | | | IDLAIDGADEVDPNLDLVK | | | |
| | | | | EKMVEAVADK | | | |
| | | | | MVEAVADK | | | |
| | | | | FIVVADDTK | | | |
| | | | | VDGDGKPYVTDNSNYIIDLYFK | | | |
| | | | | FQGVVEHGLFLGMATSVIIAGK | | | |
| | | | | NGVEVMTK | | | |
| 10 | Chlorophyll a/b | 67% | 516 | GPSGSPWYGSDR | gi\|16374 | 25036/5.12 | 0.46 ± 0.02 |
| | binding protein | | (>49) | YLGPFSGESPSYLTGEFPGDYGWDT | | | ↓ |
| | (LHCP AB 180) | | | AGLSADPETFAR | | | |
| | [Arabidopsis | | | WAMLGALGCVFPELLAR | | | |
| | thaliana] | | | FGEAVWFK | | | |
| | | | | VAGNGPLGEAEDLLYPGGSFDPLGL | | | |
| | | | | ATDPEAFAELK | | | |
| | | | | LAMFSMFGFFVQAIVTGK | | | |
| | | | | GPIENLADHLADPVNNNAWAFATN | | | |
| | | | | FVPGK | | | |
| | LHB1B2; | 31% | 376 | GPSGSPWYGSDR | gi\|18403546 | 28093/5.28 | |
| | chlorophyll | | (>49) | WAMLGALGCVFPELLAR | | | |
| | binding | | | FGEAVWFK | | | |
| | [Arabidopsis | | | LAMFSMFGFFVQAIVTGK | | | |
| | thaliana] | | | GPLENLADHLADPVNNNAWAFATN | | | |
| | | | | FVPGK | | | |
| 11 | 2-cys | 31% | 334 | AQADDLPLVGNK | gi\|9758409 | 29714/5.55 | 0.8 ± 0.04 |
| | peroxiredoxin-like | | (>41) | APDFEAEAVFDQEFIK | | | ↓ |
| | protein | | | LNTEVLGVSVDSVFSHLAWVQTDR | | | |
| | [Arabidopsis | | | SGGLGDLNYPLVSDITK | | | |
| | thaliana] | | | EGVIQHSTINNLGIGR | | | |
| 12 | PSBO-2/PSBO2; | 31% | 451 | RLTYDEIQSK | gi\|15230324 | 35226/5.92 | 2.92 ± 0.30 |
| | oxygen evolving | | (>42) | GTGTANQCPTIDGGSETFSFK | | | ↑ |
| | [Arabidopsis | | | FCFEPTSFTVK | | | |
| | thaliana] | | | VPFLFTVK | | | |
| | | | | GGSTGYDNAVALPAGGR | | | |
| | | | | NTAASVGEITLK | | | |
| | | | | SKPETGEVIGVFESLQPSDTDLGAK | | | |
| | 33 kDa oxygen- | 31% | 430 | RLTYDEIQSK | gi\|22571 | 35285/5.68 | |
| | evolving protein | | (>42) | FCFEPTSFTVK | | | |
| | [Arabidopsis | | | NAPPEFQNTK | | | |
| | thaliana] | | | VPFLFTVK | | | |
| | | | | GGSTGYDNAVALPAGGR | | | |
| | | | | GDEEELVKENVK | | | |
| | | | | NTAASVGEITLK | | | |
| | | | | SKPETGEVIGVFESLQPSDTDLGAK | | | |
| 13 | Chlorophyll a/b | 59% | 393 | GPSGSPWYGSDR | gi\|16374 | 25036/5.12 | 0.29 ± 0.03 |
| | binding protein | | (>41) | YLGPFSGESPSYLTGEFPGDYGWDTAG | | | ↓ |
| | LHCP AB 180) | | | LSADPETFAR | | | |
| | [Arabidopsis | | | WAMLGALGCVFPELLAR | | | |
| | thaliana] | | | FGEAVWFK | | | |
| | | | | VAGNGPLGEAEDLLYPGGSFDPLGLAT | | | |
| | | | | DPEAFAELK | | | |
| | | | | GPIENLADHLADPVNNNAWAFATNFVP | | | |
| | | | | GK | | | |
| | LHB1B2; | 24% | 259 | GPSGSPWYGSDR | gi\|18403546 | 28093/5.28 | |
| | chlorophyll | | (>41) | WAMLGALGCVFPELLAR | | | |
| | binding | | | FGEAVWFK | | | |
| | [Arabidopsis | | | GPLENLADHLADPVNNNAWAFATNFVP | | | |
| | thaliana] | | | GK | | | |
| 14 | Phosphopyruvate | 20% | 416 | SAVFSGASTGIYEALELR | gi\|15221107 | 51841/5.79 | 0.41 ± 0.13 |
| | hydratase | | (>42) | NQADVDALMLELDGTPNK | | | ↓ |
| | [Arabidopsis | | | IGMDVAASEFFMK | | | |
| | thaliana] | | | AAGWGVMVSHR | | | |
| | | | | SGETEDNFIADLSVGLASGQIK | | | |
| | | | | IEEELGNVR | | | |
| | | | | YAGEAFR | | | |
| 15 | TUA3 | 16% | 363 | QLFHPEQLISGK | gi\|15241168 | 50250/4.95 | 0.66 ± 0.06 |
| | [Arabidopsis | | (>41) | EDAANNFAR | | | ↓ |
| | thaliana] | | | SLDIERPTYTNLNR | | | |
| | | | | LISQIISSLTTSLR | | | |
| | | | | IHFMLSSYAPVISAAK | | | |
| | | | | DVNAAVGTIK | | | |
| 16 | ATGRP8 | 44% | 515 | CFVGGLAWATNDEDLQR | gi\|15235002 | 16626/5.58 | 2.23 ± 0.02 |
| | (GLYCINE-RICH | | (>41) | TFSQFGDVIDSK | | | ↑ |
| | PROTEIN 8); | | | GFGFVTFK | | | |
| | RNA binding/ | | | GFGFVTFKDEK | | | |
| | nucleic acid | | | VITVNEAQSR | | | |
| | binding | | | SGGGGGYSGGGGGGYSGGGGGGYER | | | |
| | [Arabidopsis | | | | | | |
| | thaliana] | | | | | | |
| 17 | Glycine-rich RNA | 53% | 581 | CFVGGLAWATDDR | gi\|21553354 | 16934/5.85 | 1.73 ± 0.15 |
| | binding protein 7 | | (>42) | ALETAFAQYGDVIDSK | | | ↑ |
| | [Arabidopsis | | | GFGFVTFK | | | |
| | thaliana] | | | SGGGGGYSGGGGSYGGGGGR | | | |
| | | | | EGGGGYGGGEGGGYGGSGGGGGW | | | |
| 18 | AT4g38970/F19H | 36% | 650 | LDSIGLENTEANR | gi\|16226653 | 43029/6.79 | 0.65 ± 0.02 |
| | 22_70 | | (>41) | TLLVSAPGLGQYVSGAILFEETLYQST | | | ↓ |
| | [Arabidopsis | | | TEGKK | | | |
| | thaliana] | | | MVDVLVEQNIVPGIK | | | |
| | | | | TAAYYQQGAR | | | |
| | | | | TVVSIPNGPSALAVK | | | |
| | | | | YAAISQDSGLVPIVEPEILLDGEHDID | | | |
| | | | | R | | | |
| | | | | ATPEQVAAYTLK | | | |
| | | | | ALQNTCLK | | | |
| | | | | YTGEGESEEAK | | | |
| 19 | RCA (RUBISCO | 22% | 620 | LVVHITK | gi\|18405145 | 52347/5.87 | 2.22 ± 0.37 |
| | ACTIVASE) | | (>42) | VPLILGIWGGK | | | ↑ |
| | [Arabidopsis | | | SFQCELVMAK | | | |
| | thaliana] | | | SFQCELVMAK | | | |
| | | | | MCCLFINDLDAGAGR | | | |
| | | | | IKDEDIVTLVDQFPGQSIDFFGALR | | | |
| | | | | LMEYGNMLVMEQENVK | | | |
| | | | | VQLAETYLSQAALGDANADAIGR | | | |
| 20 | Glutathione S- | 24% | 288 | QEAHLALNPFGQIPALEDGDLTLFESR | gi\|20197312 | 24119/6.09 | 0.55 ± 0.02 |
| | transferase (GST6) | | (>41) | GMFGMTTDPAAVQELEGK | | | ↓ |
| | [Arabidopsis | | | VLFDSRPK | | | |
| | thaliana] | | | | | | |
| 21 | Unknown protein | 10% | 169 | LSVIVAPVLR | gi\|2829916 | 29986/6.40 | 0.44 ± 0.06 |
| | [Arabidopsis | | (>41) | FADNLGDDVK | | | ↓ |
| | thaliana] | | | IENIGQPAK | | | |
| 22 | Unknown protein | 13% | 257 | SAVADNDNGESQVSDVR | gi\|6437556 | 31235/5.91 | 0.67 ± 0.03 |
| | [Arabidopsis | | (>41) | GKDPIVSGIEDK | | | ↓ |
| | thaliana] | | | LSTWTFLPK | | | |
| 23 | ABA-responsive | 48% | 487 | GVFVFDDEYVSTVAPPK | gi\|20631 | 16619/5.07 | Unique |
| | protein | | (>41) | LDAVDEANFGYNYSLVGGPGLHES | | | |
| | [Pisum sativum] | | | LEK | | | |
| | | | | VAFETIILAGSDGGSIVK | | | |
| | | | | GDAALSDAVR | | | |
| | | | | GDAALSDAVRDETK | | | |
| 24 | ABA-responsive | 39% | 250 | DADEIVPK | gi\|20631 | 16619/5.07 | Unique |
| | protein | | (>41) | EAQGVEIIEGNGGPGTIK | | | |
| | [Pisum sativum] | | | LSILEDGK | | | |
| | | | | VAFETIILAGSDGGSIVK | | | |
| | | | | GDAALSDAVR | | | |

^{a}Percentage identity between the amino-acids present in MS/MS tag and the sequences in databases.
^{b}Ion score is -10 Log (P), where P is the probability that the observed match is a random event.
Individual ion scores > value indicate identity or extensive homology (p < 0.05).
^{c}Accession number is Mascot search result using NCBI and other databases.

In all cases the Mascot searches of the NCBI non-redundant database generated significant hits with scores above the threshold value and in most cases these were a result of multiple peptide matches (Table 2). These scores are based on individual ion scores where the ion score is −10*log (P) and P is the probability that the observed match is a random event. A score above the threshold value indicates sequence identity or extensive homology (P<0.05).

Among the proteins that were identified in this study, two were unique and the identities of both were established as pea ABR17 (FIG. 7; Table 2). Although only one protein corresponding to ABR17 was expected, the fact that there are two suggests the possibility that the additional spot may be the result of post-translational modification. Most of the proteins identified in this study were associated with photosynthesis and primary metabolic pathways including putative photosystem I subunit PSI-E protein, chlorophyll binding proteins, Cp29, PSBO-2/PSBO2; oxygen evolving protein, Ribose-5-phosphate isomerase, phosphopyruvate hydratase and RUBISCO activase. Other interesting proteins identified in this study were DRT112, which is thought to be involved in DNA damage repair and glycine-rich proteins which are involved in post-transcriptional gene regulation.

Statistical Analysis

All analyses were performed using the mixed model procedure of SAS version 8e (Statistical Analysis System, 1985). Mixed model methodology was employed to perform analysis of variance and to estimate least square means and standard errors of genotype.

### Example 2

### Brassica napus (Canola)

Constitutive expression of ABR17 cDNA enhances germination under abiotic stress conditions and promotes early flowering in canola (Brassica napus)

The effect of constitutive expression of Pisum sativum ABA-responsive 17 (ABR17/PR 10.4) cDNA on DH (doubled haploid) canola (Brassica napus) line was tested to determine its effects on germination as well as other characteristics. We observed increased germination in the transgenic line in salt stress (275 mM NaCl), cold stress (5° C.), and when both the stress conditions were combined (10° C.+75 mM NaCl) as compared to the wild type (“WT”). In addition, we observed a greater rate of flowering, earlier flowering and greater height in the transgenic line when compared to the wild type at 42 days after planting. These results demonstrate that (1) ABR17 enhances germination under saline and cold conditions in canola and (2) ABR17 promotes germination and early flowering in canola when compared to the wild type. Results from this study demonstrate the utility of engineering enhanced germination under abiotic stress conditions and early flowering in a crop species. Plants with an ability to be seeded earlier, to germinate quicker, germinate in marginal soils and to flower faster will ultimately benefit agricultural production.

1. Transformation of Brassica napus

Pea ABR17 cDNA (provided by Dr. Trevor Wang, Department of Metabolic Biology, John Innes Centre, UK) was amplified and inserted into the pKLYX-71 plant expression vector and used to transform Agrobacterium tumefaciens as described by Srivastava et al. 2006. B. napus was transformed using the procedure described by Moloney et al. (1998). Embryos from T₁ seeds of successful transformants were screened on kanamycin plates (50 μg/ml) and those with a 3:1 segregation ratio were used to raise homozygous T₂ lines and subsequent seed production. The segregation ratio was determined based on the number of green to bleached embryos. Twelve independent transgenic lines contained the ABR17 construct; from these parental lines (T₁) 18 daughter lines (T₂) were selected for further screening. These 18 transgenic lines were tested for their abilities to germinate on half strength Murashige-Skoog plates (“MS plates”) (Murashige and Skoog 1962) solidified with 0.8% agarose, supplemented with 1.5% sucrose, pH 5.7, and containing 250 or 275 mM NaCl. Those with enhanced germination relative to their WT were selected for further screening.

2. Plant Growth and Germination

Seeds (WT and transgenic) were surface sterilized by soaking in 70% ethanol for 1 minute and in 20% bleach for 20 minutes after which they were rinsed four times (5 minutes each) with sterile deionized water. Surface sterilized seeds from WT and transgenic lines (3.15 and 9.5) of DH B. napus were placed on Whatman filter paper moistened with 5 ml of sterilized deionized water in Petri plates and germinated in complete darkness at room temperature, that being 21±2° C. (“RT”), in order to compare the appearance of these seedlings. For evaluating the ability of these genotypes to germinate in the presence of NaCl, lower temperatures or both, seeds were germinated and grown on half strength MS plates solidified with 0.8% agarose, supplemented with 1.5% sucrose, pH 5.7. Seeds were germinated on these MS plates in the presence or absence of 275 mM NaCl in order to assess the effects of NaCl on germination of these lines; to investigate the effects of a combination of NaCl and lower temperature stresses, plates containing either 0 or 75 mM NaCl were placed at 5° C. or 10° C. in the presence of light. For all germination experiments, seed germination was recorded daily for 7 days (at RT) or for 14 days (at 5° C. and 10° C.). Seeds were considered to have germinated if radicle emergence had occurred. In all experiments at least 5 plates per treatment and 5 seeds per plate were used and each experiment was repeated at least three times.

Seeds from the WT and transgenic lines (3.15 and 9.5) were germinated and grown under greenhouse conditions (22° C. day/18° C. night; 16 h photoperiod) for root (14 days) and whole plant observations (37 days or full maturity). For root observations, seeds were planted in Turface Athletics™ (100% calcined clay; Profile Products LLC, Ill, USA) and for whole plant observations, seeds were planted in Metro Mix® 290 (vermiculite and peat moss; Grace Horticultural Products, ON, Canada) and fertilized once every 2 weeks with 200 ppm Peters® 20-20-20. All observations were made in at least two different batches of plants planted independently and each batch consisted of at least 5 plants.

3. Expression Analysis

(a) Reverse Transcription (RT)-PCR Analysis

Total plant RNA was isolated (QIAGEN RNeasy Plant Mini Kit, Qiagen, Mississauga, ON, Canada) from pooled 5 week-old leaf tissue from WT and transgenic plants which were flash-frozen in liquid nitrogen. To ensure the complete removal of DNA prior to RT-PCR an RNase-free DNase treatment of the RNA samples was performed as per manufacturer's instructions. cDNA synthesis was performed using the iScript cDNA synthesis kit (Bio-Rad, Hercules, Calif., USA) and total RNA (50 ng) as recommended by the manufacturer. PCR reactions were carried out with 2 μl cDNA as the template and pea ABR17 using specific forward primer sequence [SEQ ID NO 3] 5′-GTGGTCGAAGCTTATGGGTGTCTTTGTTTTTGATGATGAATAC-3′ and reverse primer sequence [SEQ ID NO 4]: 5′-TATATAGCTCGAGTTAGTAACCAGGATTTGCCAAAACGTAACC-3′ using a PCR Master Mix (Promega, Md., USA) under the following thermocycling parameters: 94° C., 2 min; 35 cycles for 1 min, 94° C.; 1 min, 62° C.; 1 min, 72° C.; and an extension for 10 min, 72° C. Plant 18s rRNA was used in these experiments as internal control using forward primer sequence:[SEQ ID NO 7]: 5′-CCAGGTCCAGACATAGTAAG-3′ and reverse primer sequence [SEQ ID NO 8]:5′-GTACAAAGGGCAGGGACGTA-3′, these primers being specific for this gene (Duval et al. 2002). Amplification products were separated on a 1% agarose gel and visualized under UV light after staining with ethidium bromide.

(b) Quantitative Real-Time-PCR (qRT-PCR) Analysis

Quantitative real-time polymerase chain reaction (qRT-PCR) was performed to investigate the relative levels of expression of ABR17 using the expression of actin gene as the internal control. The sequences of the ABR17-specific primers and probes were as follows. The forward primer sequence was 5′-AAATGGAGGTCCAGGAACCAT-3′ [SEQ ID NO 9] and the reverse primer sequence was 5′-AGCACATAGTTGGTTTTTCCATCTT-3′ [SEQ ID NO 10]. The probe sequence was 5′-AGAAGCTATCCATTCTT-3′ [SEQ ID NO 11]. For the amplification of the actin gene, the following primers and probes were used. The forward primer sequence was 5′-GCCATTCAGGCCGTTCTTT-3′ [SEQ ID NO 12] and the reverse primer sequence was 5′-ATCGAGCACAATACCGGTTGT-3′ SEQ ID NO 13]. The probe sequence was 5′-TCTATGCCAGTGGTCG-3′ [SEQ ID NO 14].

qRT-PCR primer sets were designed using Primer Premier software (Applied Biosystems Inc., CA, USA) to generate amplification products that were approximately 70-80 bp in size. RNA isolation and cDNA preparation was performed as described above. PCR reactions contained 2 μl of cDNA (5× dilution), 5 ρmol of probe, 22.5 ρmol of each primer and 1× TaqMan UniversPCR Master Mix (Roche, N.J., USA). The SNP RT template program was used for real-time quantification and was performed in an ABI prism 7700 Sequence detector (Applied Biosystems). The delta-delta method was used to determine relative expression using the following formula: Relative Expression=2−[^{ΔCt sample-ΔCt control}], where Ct is the threshold cycle (Livak and Schmittgen 2001). The relative expression of ABR17 in the transgenic lines was normalized against expression in the WT which was considered to be 1. The experiment was repeated at least three times.

(c) Western Blot Analysis

Pooled two week-old B. napus seedlings (0.2 g) were crushed in liquid N₂ and 500 μl extraction buffer (0.5 M Tris-HCl, pH 6.8; containing 10% glycerol; 10% SDS and 60 mM DTT) was added. The tubes were vortexed and then boiled for 5 minutes in a water bath. After cooling, a few crystals of protamine sulfate were added and the tubes were incubated at RT for 15 min and then centrifuged at 12,500 g for 15 min. Ice-cold acetone containing 0.07% DTT was added to the supernatant and the tubes were centrifuged as described above. The pellets were vacuum dried for 15 min and resuspended in 150 μl of 50 mM Tris pH 6.8 containing 0.5% SDS and centrifuged a final time as above. The concentration of protein in the samples was determined using a modified Bradford assay and the samples stored at −20° C.

Proteins (25 μg) were separated by SDS-polyacrylamide gel (15%) electrophoresis (Laemmli 1970) using a vertical slab system (Mini PROTEAN 3, Bio-Rad) at constant 150 V until the dye front reached the bottom of the gel. After electrophoresis, the gel was placed in transfer buffer (48 mM Tris, pH 9.2 containing 39 mM glycine, 20% methanol and 1.3 mM SDS) for 15 min and subsequently transferred to polyvinylidene difluoride (PVDF) membrane for 25 min at 15 V using Trans Blot SD, semi-dry transfer apparatus (Bio-Rad). The PVDF membrane was incubated with TBS (10 mM Tris-HCl, pH 7.5; 150 mM NaCl) containing 5% non-fat skim milk powder. The membrane was subsequently blocked for 2 hours and then rinsed with TTBS (TBS containing 0.05% Tween 20) for 10 min. The membrane was then incubated for one hour in the primary antibody solution (rabbit anti-ABR17; Srivastava et al. 2006) diluted 1:20 000 in TTBS. The membrane was washed 3 times for 5 minutes each with TTBS after which the membrane was incubated for 1 hour in with the secondary antibody (goat anti-rabbit immunoglobulin G-horseradish peroxidase conjugate (Abcam, Mass., USA) which was diluted 1:10 000 in TTBS. After this incubation, the membrane was washed as described earlier, followed by a 5 minutes final wash with TBS. Immunoreactive bands on the membrane were visualized by staining with TMB peroxidase substrate kit (Vector Laboratories Inc., CA, USA) according to manufacturer's instructions.

4. Statistical Analysis

For statistical analysis, analysis of variance was conducted by using proc mixed SAS (version 8e, Statistical Analysis System 1985) where genotype was considered as fixed effect and replicate was considered as random effect for all plant characteristics (germination, root and shoot length, height and flowering). The values presented on the graphs are based on the LS means estimates. Since the transgene had a significant effect on plant height and flowering we hypothesized that the early flowering observed in the transgenic plants might be due to the effect of height. In order to test this hypothesis we conducted an analysis of covariance on flowering as dependent variable with proc mixed (SAS) where genotype was considered as fixed effect, replicate was considered as random effect and height was considered a covariant.

Results

1. Generation of Transgenic Brassica napus and Confirmation of Gene Expression

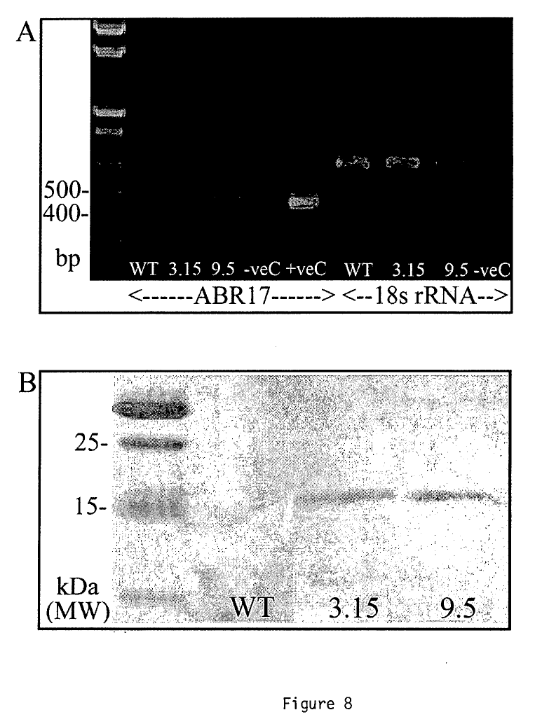

Transgenic DH B. napus plants containing one copy of ABR17 were chosen based on their segregation ratios on plates containing kanamycin. Seeds from 18 transgenic lines containing a single copy of the cDNA were screened on half strength MS media containing 250 mM NaCl. Two lines, showing the best relative germination (lines 3.15 and 9.5), were selected for further studies and homozygous T₂ seeds were used for all experiments. Transgene expression was confirmed using reverse transcriptase-polymerase chain reaction (RT-PCR) with pea ABR17-specific primers with 18s rRNA gene as the internal control. FIG. 8A shows analysis of ABR17 and 18S RNA expression by RT-PCR. As shown in FIG. 8A, the 417 bp amplification product of ABR17 verified expression in the transgenic lines (3.15 and 9.5) and was absent in the WT whereas the amplification of the 18s rRNA gene was present in both WT and transgenic plants(visualized by staining with ethidium bromide). FIG. 8B shows analysis of protein extracts from two-week old seedlings by Western Blot. As shown in FIG. 8B, the presence of pea ABR17 protein was also confirmed in these transgenic plants by Western blot analysis which revealed an immunoreactive band at the expected molecular weight (˜16-17 kDa) in both transgenic lines (3.15 and 9.5) but not in the WT. Results from these experiments confirmed that the pea ABR17 cDNA is integrated, transcribed and translated in B. napus.

2. Appearance of ABR17 Transgenic B. napus Seedlings and Plants

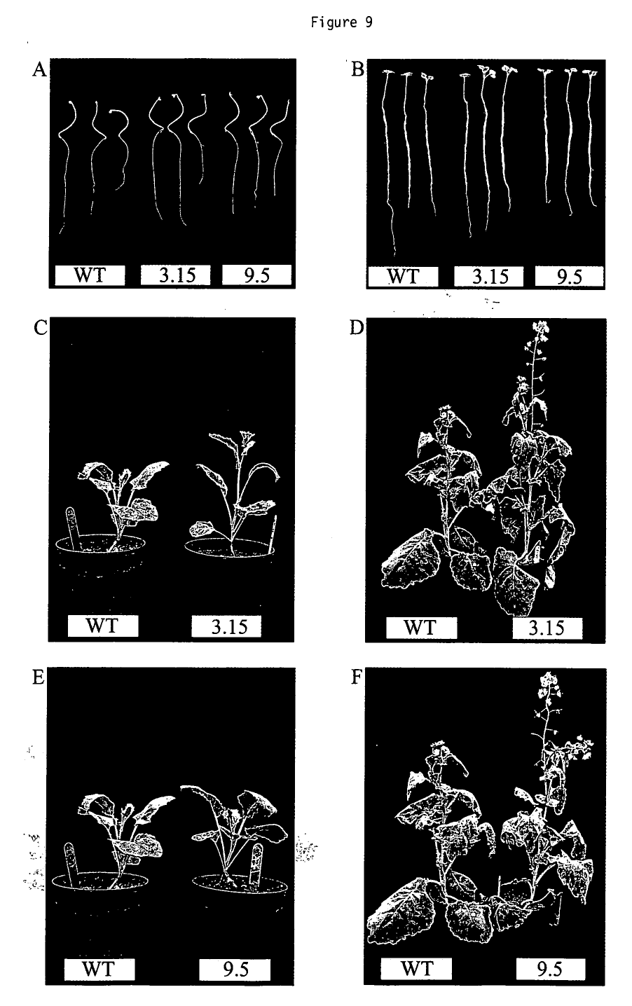

FIG. 9 shows the appearance of WT and ABR17 transgenic B. napus seedlings and plants. Appearance of (A) 7-day, (B) 14 day- and 37 day-old seedlings (C and E). The appearance of 42-day old plants demonstrating early flowering in the transgenic lines is shown in panels D and F.

As shown in FIGS. 9A and 9B, the appearance of WT and transgenic seedlings (3.15 and 9.5) grown on filter paper in sterile water for 7 days and in clay growth medium for 14 days did not demonstrate any visible difference at these stages. Within the WT and transgenic lines there was a variation in root length and shoot length at both stages; however, there was no distinct difference between the WT and transgenic lines. As shown in FIGS. 9C, 9D and 9F, at 37 days after planting (DAP) plants grown in soil under greenhouse conditions started exhibiting differences between the WT and transgenic line. Transgenic line 3.15 had a longer internode length and more advanced floral bud formation when compared to the WT. Transgenic line 9.5 did not demonstrate any difference from its WT at this stage. As shown in FIGS. 9D and 9F, at 42 DAP there was a visual difference between the WT and transgenic lines 3.15 and 9.5. Both transgenic lines were developmentally ahead of the WT and had a greater number of flowers per plant.

3. Effects of Salinity and Low Temperature Stresses on Germination of ABR17-Transgenic Brassica napus

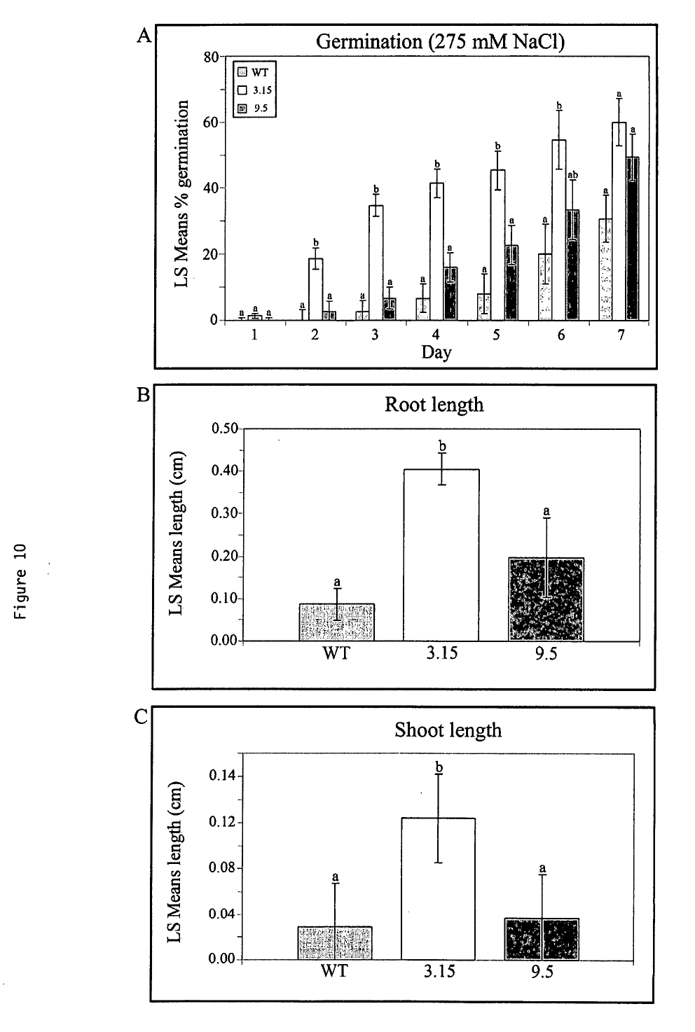

The ability of the transgenic lines to germinate in the presence of 275 mM NaCl was evaluated at room temperature (RT; 21±2° C.) and the results are presented in FIG. 10. Specifically FIG. 10 shows thee effect of NaCl on germination, root length and shoot length of WT and ABR17 transgenic B. napus seedlings. FIG. 10A shows the effect of 275 mM NaCl on germination. FIG. 10B shows root length (cm). FIG. 10C shows shoot length (cm) of 7 day-old seedlings. The transgenic line 3.15 demonstrated significantly (P<0.05) higher percent germination on all days except days 1 and 7 with the germination on day 3 the being more that 25% higher in the transgenic line 3.15 compared to the WT (FIG. 10A). Although not significant, on day 1 line 3.15 had started germinating while lines 9.5 and the WT had not. Furthermore, line 3.15 showed significantly longer shoot and root length when compared to the WT and line 9.5, indicating that germination as well as initial emergence and growth was greater in line 3.15 during the first seven days compared to the other lines (FIGS. 10B and 10C).

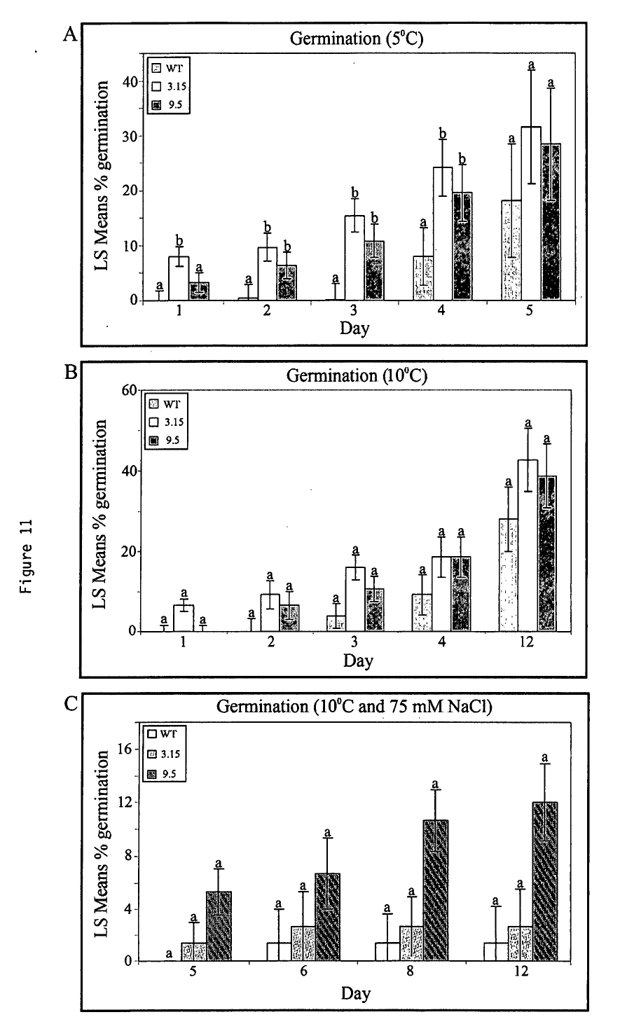

The ability of ABR17-transgenic B. napus and WT seeds was evaluated for germination at 10° C. and 5° C. alone as well as in combination with these low temperature stresses and salinity. FIG. 11 shows the effect of low temperature and NaCl on the germination of WT and ABR17 transgenic B. napus seedlings. FIG. 11A shows the effect of 5° C. FIG. 11B shows the effect of 10° C. FIG. 11C shows the effect of 10° C. plus 75 mM NaCl on the germination of the WT and transgenic lines 3.15 and 9.5. Once again, at 5° C., line 3.15 showed significantly greater germination than WT on day 1 (FIG. 11A). Although line 9.5 was not significantly ahead of the WT at this time, it had started germinating while the WT had not. By day two the trend in greater germination demonstrated by line 9.5 on day 1 had become significant. On day 2, 3, and 4 both lines 3.15 and 9.5 were significantly ahead of the WT. In fact, by the third day, transgenic lines 3.15 and 9.5 demonstrated between 5 and 10% greater germination, respectively, than the other lines tested. At 10° C., although there were no significant differences in percent germination between the two transgenic lines that exhibited higher germination at 5° C. (3.15 and 9.5), the transgenic line 3.15 displayed a similar trend of increased germination on days 1-3 (FIG. 11B). In addition to investigating the ability of ABR17-transgenic seeds to germinate at the lower temperatures described above, we also investigated the effects of a combination of lower temperature and NaCl on the ability of these lines to germinate by testing the effect of 75 mM NaCl on the ability of B. napus to germinate at 10° C. Under these conditions, significantly more seeds of the transgenic line 9.5 germinated than the WT on days 8 and 12 (FIG. 11C).

Salt tolerance in the Brassicas is often associated with ion exclusion. Salt exposed B. napus plants have greater K⁺/Na⁺ ratios in their tissues indicating that they are able to preferentially accumulate K⁺ over Na⁺, avoiding much of the nutritional stress associated with increased Na⁺ uptake under saline conditions. The documented tolerance exhibited by B. napus (Francois 1994; Steppuhn and Raney, 2005) was evident in the DH line we were testing, as salinity tolerance during germination (radicle emergence) was demonstrated up to 200 mM NaCl in the WT and both transgenic lines 3.15, and 9.5 constitutively expressing ABR17 (data not shown). However, line 3.15 demonstrated increased germination at 275 mM NaCl when compared to the WT. Generally, low levels of salinity will slow germination down, but not affect the final number of seedlings that germinate, while at higher levels of salinity, the speed of germination will be further reduced with overall germination percentage also decreasing (Bernstein and Hayward 1958). The WT and the transgenic line 9.5 demonstrated reduced germination, while the high performing transgenic line 3.15 was ahead in germination after 48 h and continued to be ahead for the first five days after seeding. The increased germination at 275 mM NaCl indicates that the transgenic line does show enhanced germination under saline conditions. Although root growth does not show the effects of salt stress as much as shoot growth (Munns 2002), line 3.15 had significantly greater root growth than line 9.5 and the WT. Shoot growth in transgenic line 3.15 was also significantly longer than the WT. The greater root length in 3.15 may give this line the added benefit that rapidly dividing root cells have in providing more area for salt sequestration (Bartels and Sunkar 2005) limiting the concentration of salt in the plant (Munns 2002). Furthermore, as the seedlings mature a higher root to shoot ratio ultimately results in better exploitation of soil resources (Pasternak 1987).

Although seedling stages are often regarded as the most salt sensitive growth stage, Bernstein and Hayward (1958) state that the poor emergence in saline soils may be a result of the soil in the upper portion of the field being more saline, due to evapotranspiration, field morphology and low moisture, rather than low tolerance. Theoretically, in the same soil, the adult plant could avoid the saline regions of the soil profile with their more extensive root system and appear more tolerant. As a result, using one stage of growth to determine the salt tolerance of a specific crop may not be the best approach in determining if a plant is indeed salt tolerant. The early germination seen in our transgenic lines is important, because as Steppuhn and Raney (2005) demonstrated germination and early seedling growth are important parameters in determining the future success of canola under saline soil conditions. They demonstrated that the canola varieties used in their study were comparable to salt tolerant Harrington barley if they displayed good emergence (Steppuhn and Raney 2005). It is important to determine salt tolerance during early growth stages, so that we can establish initial salt tolerance; however, it will be equally important in future studies to evaluate the performance of the transgenic line as it matures under saline conditions.

Low temperature stress, like salinity stress, impacts crop productivity. Cold temperatures during germination can delay the onset of germination and reduce crop yields; however, the mechanism of how low temperature impacts germination is poorly understood (Salaita et al. 2005). There have been numerous germination studies on impact of low temperature stress in tomato (reviewed in Salatia et al. 2005), Arabidopsis (Srivastava et al. 2006, Salaita et al. 2005) and other plants. Cold temperature studies have been performed on Arabidopsis seedlings and a number of COR genes have been found to be involved in cold tolerance that encode Late Embryo Abundant (LEA)-like proteins (reviewed in Mahajan and Tuteja 2005) which are synthesized in the seedlings in response to dehydration stress. The promoter elements of COR genes contain DRE (dehydration responsive elements) or CRT (C-repeats) and some of them contain ABRE (ABA-responsive element) (Stockinger 1997; Yamaguchi-Shinozaki 1994). The ability to germinate under cold conditions is important in the northern hemisphere, because cold temperatures in the spring may negatively affect crop germination, early seedling growth, and ultimately, stand establishment. Furthermore, the short growing season and moisture limitations later in the growing season, particularly in the prairie regions, make it important to seed canola crops early so they are able to reach maturity without experiencing a reduction in yield (Agandi et al. 2004). Enhanced germination under salt and cold due to overexpression of ABR17 cDNA may indicate that molecular responses common to both stresses are responsible for the observed effects mediated by ABR17. Our observation that the constitutive expression of pea ABR17 cDNA in B. napus enhances its germination under cold as well as saline conditions may have utility in the genetic engineering of crops.

4. ABR17 Expression Leads to Early Flowering in B. napus

The transgenic lines tested in this study exhibited dramatic differences in development when visually compared to the WT at 42 DAP, as can be seen in FIGS. 9D and 9F, specifically with regard to number of plants flowering and the amount of flowers per plant.

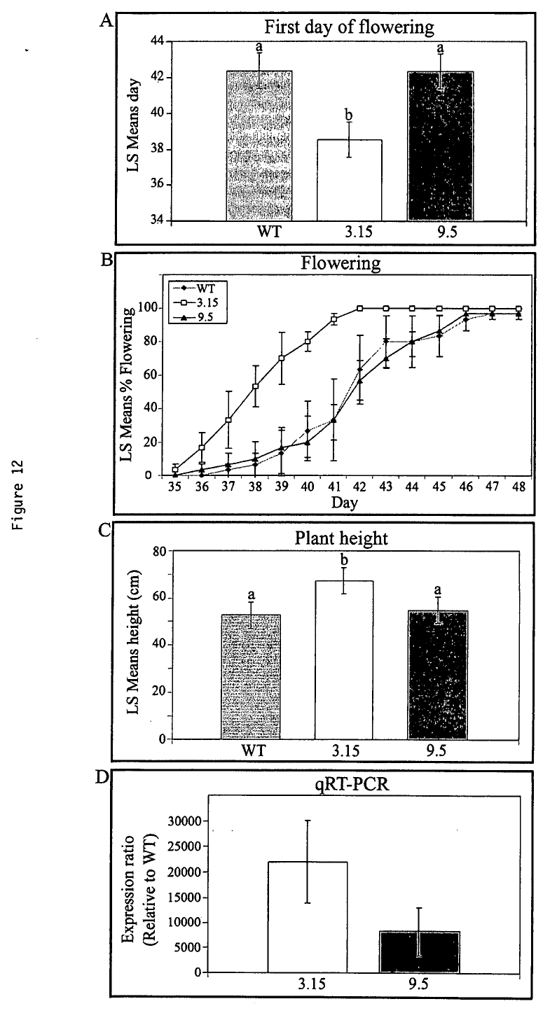

FIG. 12 shows flowering and height of ABR17 transgenic and WT B. napus adult plants 42 days after planting (DAP). FIG. 12A shows first day of flowering. FIG. 12B shows rate of flowering. FIG. 12C shows plant height of ABR17 transgenic and WT B. napus adult plants. FIG. 12D shows the relative expression of ABR17 in the transgenic lines (3.15 and 9.5). The transgenic line 3.15 flowered significantly (P<0.05) earlier when compared to the WT and the transgenic line 9.5 (FIG. 12A). Line 3.15 also displayed a significantly faster rate of plants flowering per day when compared to line 9.5 and the WT (FIG. 12B). Furthermore, line 3.15 at 42 DAP was significantly taller than the WT (FIG. 12C). When the effect of height was removed from the analysis it was apparent that the height did not contribute to the difference in flowering time observed between the WT and line 3.15. A possible reason for the lack of significant early flowering by the transgenic line 9.5 could be that the transgene was integrated in different locations on the B. napus genome leading to differential expression which affected its ability to flower earlier.

Similar to germination, flowering is an important developmental process that contributes to determining crop productivity. The timing of flowering becomes extremely important in northern latitudes because of the shorter growing season, which reduces the time for reproductive growth (Chandler et al. 2005). This can be the case in Alberta, especially the Peace Region where the growing season is short, and in the southern part of the province where adequate precipitation is a problem. For example, if the plant switches from vegetative to reproductive growth too early seed production can be limited, because the plants do not have enough leaves and roots to provide energy to the developing flowers and seeds. On the other hand, if the switch to reproduction is delayed, even though the plant is large enough to gather photosynthates and nutrients, it may not have enough time to produce mature seeds (Franke et al. 2006). Numerous studies on the transition from reproductive growth to flowering have been undertaken (reviewed in Bernier and Perilleux 2005). Aside from environmental cues, like photoperiod and vernalization, cytokinins (CK) and gibberellins (GA) also affect the transition to flowering. Application of both phytohormones on the shoot apical meristem (SAM) of Sinapsis alba activate the SaAMDS A gene that may be involved in the transition to flowering (Bonhomme et al. 2000). For example, Chaudhury et al. (1993) found that Arabidopsis amp1 mutant with high CK levels also demonstrated early flowering. Furthermore, Dewitte et al. (1999) reported that organ formation, including flowering is correlated with increased endogenous CK levels in tobacco. Indeed, we have demonstrated previously (Srivastava et al. 2006) that transgenic A. thaliana plants expressing pea ABR17 cDNA flower earlier and that this may be the result of enhanced, endogenous CK levels in that species (Srivastava et al. 2007). The earlier flowering observed in ABR17-transgenic lines investigated in this study may also be the result of enhanced CK levels although this suggestion must be verified through the determination of endogenous CKs in these lines.

5. Relative Expression of ABR17 cDNA in Transgenic Lines

Even though RT-PCR experiments revealed the expression of the pea ABR17 cDNA, the relative levels of expression in these lines could not be ascertained using that technique. In order to determine whether differences in the relative levels of expression between the two transgenic lines were contributing to the observed differences in germination as well as flowering, we performed qRT-PCR experiments. These experiments revealed that line 3.15 had the highest level of ABR17 expression when compared to line 9.5 as seen in FIG. 12D. These results correlate well with the observed responses of these lines with respect to germination as well as earlier flowering. For example, the transgenic line 3.15 was observed to be the best when tested for germination in the presence of 275 mM NaCl, had the best developed roots and shoots (FIGS. 10B and 10C) and flowered earlier (FIGS. 9D and 12A) compared to line 9.5. The excellent correlation observed between the levels of ABR17 expression, which was approximately 2-fold higher in line 3.15 (FIG. 12D), and the phenotypic characteristics of this line clearly indicate that the observed phenomena are the result of the transgene expression. The differences between the two transgenic lines with respect to the relative levels of expression may be the result of differences in the location of transgene integration i.e. positional effects.

While the invention has been described in conjunction with the disclosed embodiments, it will be understood that the invention is not intended to be limited to these embodiments. On the contrary, the invention is intended to cover alternatives, modifications and equivalents, which may be included within the spirit and scope of the invention as defined by the appended claims. Various modifications will remain readily apparent to those skilled in the art.

### REFERENCES

The following references are cited in the application at the relevant portion of the application. Each of these references is incorporated herein by reference.

Abbasi, F. M. and Komatsu, S. (2004) A proteomic approach to analyze salt-responsive proteins in rice leaf sheath. Proteomics 4, 2072-2081.

Adelman J P et al. (1983) In vitro deletional mutagenesis for bacterial production of the 20,000-dalton form of human pituitary growth hormone. DNA 2(3), 183-93.

Agrawal, G. K., Yonekura, M., Iwahashi, Y., Iwahashi, H. and Rakwal, R. (2005) System, trends and perspectives of proteomics in dicot plants. Part III: Unraveling the proteomes influenced by the environment, and at the levels of function and genetic relationships. J. Chromatogr. B Analyt. Technol. Biomed. Life Sci. 815, 137-145.

Angadi S V, Cutforth H W, McConkey B G, Gan Y (2004) Early seeding improves the sustainability of canola and mustard production the Canadian semiarid prairie. Can J Plant Sci 84: 705-711.

Apse, M. P., Aharon, G. S., Snedden, W. A. and Blumwald, E. (1999) Salt tolerance conferred by overexpression of a vacuolar Na+/H+ antiport in Arabidopsis. Science, 285, 1256-1258.

Ausubel et al. (1987) Current Protocols in Molecular Biology, Greene Publishing Assoc. and Wiley Interscience

Ausubel et al. (2005) Current Protocols in Molecular Biology, John Wiley & Sons Inc; ringbound edition.

Balmer, Y., Vensel, W. H., Tanaka, C. K., Hurkman, W. J., Gelhaye, E., Rouhier, N., Jacquot, J. P., Manieri, W., Schurmann, P., Droux, M. and Buchanan, B. B. (2004) Thioredoxin links redox to the regulation of fundamental processes of plant mitochondria. Proc. Natl. Acad. Sci. USA. 24, 2642-2647.

Bantignies, B., Seguin, J., Muzac, I., Dedaldechamp, F., Gulick, P. and Ibrahim, R. (2000). Direct evidence for ribonucleolytic activity of a PR-10-like protein from white lupin roots. Plant Mol. Biol. 42, 871-881.

Bartels D, Sunkar R (2005) Drought and salt tolerance in plants. Crit Rev Plant Sci 24: 23-58.

Bernstein L, Hayward H E (1958) Physiology of salt tolerance. Annu Rev Plant Physiol 9: 25-46.

Bernier G, Perilleux C (2005) A physiological overview of the genetics of flowering time. Plant Biotechnol J 3: 3-16.

Biesiadka, J., Bujacz, G., Sikorski, M. M. and Jaskolski, M. (2002) Crystal structures of two homologous pathogenesis-related proteins from yellow lupine. J. Mol. Biol. 319, 1223-1234.

Bonhomme F, Kurz B, Melzer S, Bernier G, Jacqmard A (2000) Cytokinin and gibberellin activate SaMADS A, a gene apparently involved in regulation of the floral transition in Sinapis alba. Plant J 24: 103-111.

Borsics, T. and Lados, M. (2002) Dodder infection induces the expression of a pathogenesis-related gene of the family PR-10 in alfalfa. J. Exp. Bot. 53, 1831-1832.

Boyer, J. S. (1982) Plant productivity and environment. Science, 218, 443-448.

Bray, E. A., Baiely-Serre, J. and Weretilnyk, E. (2000) Responses to abiotic stresses. In: Biochemistry and Molecular Biology of Plants (Grusissem, W., Buchannan, B. and Jones, R., eds), pp. 1158-1249. American Society of Plant Physiologists: Rockville, Md.

Breiteneder, H., Pettenburger, K., Bito, A., Valenta, R., Kraft, D., Rumpold, H., Scheiner, O. and Breitenbach. M. (1989) The gene coding for the major birch pollen allergen Betv1, is highly homologous to a pea disease resistance response gene. EMBO J. 8, 1935-1938.

Bufe, A., Spangfort, M. D., Kahlert, H., Schlaak, M. and Becker, W. M. (1996) The major birch pollen allergen, Bet v 1, shows ribonuclease activity. Planta, 199, 413-415.

Chandler J, Corbesier L, Spielmann P, Dettendorfer J, Stahl D, Apel K, Melzer S (2005) Modulating flowering time and prevention of pod shatter in oilseed rape. Mol Breeding 15: 87-94.

Chaudhury A M, Letham S, Craig S, Dennis E S (1993) amp1—a mutant with high cytokinin levels and altered embryonic pattern, faster vegetative growth, constitutive photomorphogenesis and precious flowering. Plant J 4: 907-916.

Chinnusamy, V., Schumaker, K. and Zhu, J. K. (2004) Molecular genetic perspectives on cross-talk and specificity in abiotic stress signalling in plants. J. Exp. Bot. 55, 225-236.

Close, T. J., Fenton, R. D., Yang, A., Asghar, R., DeMason, D. A., Crone, D. E., Meyer, N. C., and Moonan, F. (1993) Dehydrin: The protein. In: Plant Responses to Cellular Dehydration during Environmental Stress (Close, T. J. and Bray, E. A., eds), pp. 104-118. Rockville, Md.: American Society of Plant Physiologists).

Clough, S. and Bent, A. (1998) Floral dip: a simplified method for Agrobacterium-mediated transformation of Arabidopsis thaliana. Plant J. 16, 735-743.

Colditz, F., Nyamsuren, O., Niehaus, K., Eubel, H., Braun, H. P. and Krajinski, F. (2004) Proteomic approach: identification of Medicago truncatula proteins induced in roots after infection with the pathogenic oomycete Aphanomyces euteiches. Plant Mol. Biol. 55, 109-120.

Constabel, C. P. and Brisson, N. (1992) The defence-related STH—2 gene product of potato shows race-specific accumulation after inoculation with low concentrations of Phytophihora infestans zoospores. Planta, 188, 289-295.

Coursolle, C., Bigras, F. J. and Margolis, H. A. (1998) Frost tolerance and hardening capacity during the germination and early developmental stages of four white spruce (Picea glauca) provenances. Can. J. Bot. 76, 122-129.

Crowell, D. N., John, M. E., Russell, D. and Amasino, R. M. (1992) Characterization of a stress-induced, developmentally regulated gene family from soybean. Plant Mol. Biol. 18, 459-466.

Dewitte W, Chiappetta A, Azmi A, Witters E, Strnad M, Rembur J, Noin M, Chriqui D, Van Onckelen H (1999) Dynamics of cytokinins in apical shoot meristems of a day-neutral tobacco during floral transition and flower formation. Plant Physiol 119: 111-121.

Duval, F. D., Renard, M., Jaquinod, M., Biou, V., Montrichard, F. and Macherel, D. (2002) Differential expression and functional analysis of three calmodulin isoforms in germinating pea (Pisum sativum L.) seeds. Plant J. 32, 481-493.

Francois L E (1994) Growth, seed yield, and oil content of canola grown under saline conditions. Agronomy J 86: 233-237.

Franke D F, Ellis A G, Dharjwa M, Freshwater M, Fujikawa M, Padron A, Weis A E (2006) A steep cline in flowering time for Brassica rapa in southern California: population-level variation in the field and the greenhouse. Int J Plant Sci 167: 83-92.

Fristensky, B., Riggleman, R. C., Wagoner, W. and Hadwiger, L. A. (1985) Gene expression in susceptible and disease resistant interactions of pea induced with Fusarium solani pathogens and chitosan. Physiol. Plant Pathol. 27, 15-28.

Fristensky, B., Horovitz, D. and Hadwiger, L. A. (1988) cDNA sequence for pea disease resistance response genes. Plant Mol. Biol. 11, 713-715.

Goday, A., Jensen, A. B., Culianez-Macia, F. A., Mar Alba, M., Figueras, M., Serratosa, J., Torrent, M. and Pages, M. (1994) The maize abscisic acid-responsive protein Rab17 is located in the nucleus and interacts with nuclear localization signals. Plant Cell, 6, 351-360.

Guiltinan, M. J., Marcotte, W. R. and Quatrano, R. S. (1990) A plant leucine zipper protein that recognizes an abscisic acid response element. Science, 250, 267-271.

Haake, V., Cook, D., Riechmann, J. L., Pineda, O., Thomashow, M. F. and Zhang, J. Z. (2002) Transcription factor CBF4 is a regulator of drought adaptation in Arabidopsis. Plant Physiol. 130, 639-648.

Hasegawa, P. M., Bressan, R. A., Zhu, J-K. and Bohnert, H. J. (2000) Plant cellular and molecular responses to high salinity. Annu. Rev. Plant Physiol. Plant Mol. Biol. 51, 463-499.

Hashimoto, M., Kisseleva, L., Sawa, S., Furukawa, T., Komatsu, S. and Koshiba, T. (2004) Novel rice PR10 protein, RSOsPR10, specifically induced in roots by biotic and abiotic stresses, possibly via the jasmonic acid signaling pathway. Plant Cell Physiol. 45, 550-559.

Heide, H., Kalisz, H. M. and Follmann, H. (2004) The oxygen evolving enhancer protein 1 (OEE) of photosystem II in green algae exhibits thioredoxin activity. J. Plant Physiol. 161, 139-149.

Imai, R., Chang, L., Ohta, A., Bray, E. A. and Takagi, M. (1996) A lea-class gene of tomato confers salt and freezing tolerance when expressed in Saccharomyces cerevisiae. Gene, 170, 243-248.

Iturriaga, E. A., Leech, M. J., Barratt, D. H. P., Wang, T. L. (1994) Two ABA-responsive proteins from pea (Pisum sativum L.) are closely related to intracellular pathogenesis-related proteins. Plant Mol. Biol. 24, 235-240.

Jaglo-Ottosen, K. R., Gilmour, S. J., Zarka, D. G., Schabenberger, O., Thomashow, M. F. (1998) Arabidopsis CBF1 overexpression induces COR genes and enhances freezing tolerance. Science, 280, 104-106.

James, H. E., Bartling, D., Musgrove, J. E., Kirwin, P. M., Herrmann, R. G. and Robinson, C. (1989) Transport of proteins into chloroplasts. Import and maturation of precursors to the 33-, 23-, and 16-kDa proteins of the photosynthetic oxygen-evolving complex. J. Biol. Chem. 264, 19573-19576.

Jensen, P. E., Haldrup, A., Rosgaard, L. and Scheller, H. V. (2003) Molecular dissection of photosystem I in higher plants: topology, structure and function. Physiologia Plantarum. 119, 313-321.

Kang, J. Y., Choi, H. I., Im, M. Y. and Kim, S. Y. (2002) Arabidopsis basic leucine zipper proteins that mediate stress-responsive abscisic acid signaling. Plant Cell, 14, 343-357.

Kasuga, M., Liu, Q., Miura, S., Yamaguchi-Shinozaki, K. and Shinozaki, K. (1999) Improving plant drought, salt, and freezing tolerance by gene transfer of a single stress-inducible transcription factor. Nature Biotech. 17, 287-291.

Kasten, B., Buck, F., Nuske, J. and Reski, R. (1997) Cytokinin affects nuclear- and plastome-encoded energy-converting plastid enzymes. Planta. 201, 261-272.

Kav, N. N. V., Srivastava, S., Goonewardene, L. and Blade, S. F. (2004) Proteome-level changes in the roots of Pisum sativum L. in response to salinity. Ann. Appl. Biol. 145, 217-230.

Kim, J-B., Kang, J-Y. and Kim, S. Y. (2004) Over-expression of a transcription factor regulating ABA-responsive gene expression confers multiple stress tolerance. Plant Biotech. J. 2, 459-466.

Kim, Y. O., Kim, J. S. and Kang, H. (2005) Cold-inducible zinc finger-containing glycine-rich RNA-binding protein contributes to the enhancement of freezing tolerance in Arabidopsis thaliana. Plant J. 42, 890-900.

Kirk, J. T. O. (1968) Studies on the dependence of chlorophyll synthesis on protein synthesis in Euglena gracilis, together with a nomogram for determination of chlorophyll concentration. Planta (Berl.), 78, 200-207.

Kirk, J. T. O. and Allen, R. L. (1965) Dependence of chloroplast pigment synthesis on protein synthesis: effect of actidione. Biochem. Biophys. Res. Comm. 21, 523-530.

Laemmli, U. K. (1970) Cleavage of structural proteins during the assembly of the head of bacteriophage T4. Nature 227, 680-685.

Lee, H., Guo, Y., Ohta, M., Xiong, L., Stevenson, B. and Zhu, J. K. (2002) LOS2, a genetic locus required for cold-responsive gene transcription encodes a bi-functional enolase. EMBO J. 21, 2692-2702.

Leung, J. and Giraudat, J. (1998) Abscisic acid signal transduction. Annu. Rev. Plant Biol. 49, 199-222.

Lewin B, (2000) Genes VII, published by Oxford University Press.

Livak K J, Schmittgen T D (2001) Analysis of relative gene expression data using real-time quantitative PCR and the 2 (T)(-Delta Delta C) method. Methods 25: 402-408.

Mahajan S, Tuteja N (2005) Cold, salinity, and drought stresses: an overview. Arch Biochem Biophy 444: 139-158.

Maniatis T, Fritsch E F, and Sambrook J (1989) Molecular Cloning: A Laboratory Manual, Cold Spring Harbor Laboratory, Cold Spring Harbor (N.Y.)

Meyers R A (ed.) (1995) Molecular Biology and Biotechnology, a Comprehensive Desk Reference, published by VCH Publishers, Inc.

McCarty, D. R. (1995) Genetic control and integration of maturation and germination pathways in seed development. Annu. Rev. Plant Physiol. Plant Mol. Biol. 46, 71-93.

McGee, J. D., Hamer, J. E. and Hodges, T. K. (2001) Characterization of a PR-10 pathogenesis-related gene family induced in rice during infection with Magnaporthe grisea. Mol. Plant Microbe Interact. 14, 877-886.

Moiseyev, G. P., Beintema, J. J., Fedoreyeva, L. I. and Yakovlev, G. I. (1994) High sequence similarity between a ribonuclease from ginseng calluses and fungus-elicited proteins from parsley indicates that intracellular pathogenesis-related proteins are ribonucleases. Planta. 193, 470-472.

Moloney M M, Walker J M, Sharma K K (1989) High-efficiency transformation of Brassica napus using Agrobacterium vectors Plant Cell Rep 8: 238-242.

Munns R (2002) Comparative physiology of salt and water stress. Plant Cell Environ 25: 239-250.

Murashige T, Skoog F (1962) A revised medium for rapid growth and bio assays with tobacco tissue cultures. Physiol Plantarum 15: 473-497.

Ndong, C., Danyluk, J., Wilson, K. E., Pocock, T., Huner, N. P. A. and Sarhan, F. (2002) Cold-regulated cereal chloroplast late embryogenesis abundant-like proteins. Molecular characterization and functional analyses. Plant Physiol. 129, 1368-1381.

Neuwald, A. F., Aravind, L., Spouge, J. L. and Koonin, E. V. (1999) AAA+: A class of chaperone-like ATPases associated with the assembly, operation, and disassembly of protein complexes. Genome Res. 9, 27-43.

Oh, S. J., Song, S. I., Kim, Y. S., Jang, H. J., Kim, S. Y., Kim, M., Kim, Y. K., Nahm, B. H. and Kim, J. K. (2005) Arabidopsis CBF3/DREB1A and ABF3 in transgenic rice increased tolerance to abiotic stress without stunting growth. Plant Physiol. 138, 341-351.

Osmark, P., Boyle, B. and Brisson, N. (1998) Sequential and structural homology between intracellular pathogenesis-related proteins and a group of latex proteins. Plant Mol. Biol. 38, 1243-1246.

Park C. J., Kim K. J., Shin R., Park J. M., Shin Y. C. and Paek K. H. (2004) Pathogenesis-related protein 10 isolated from hot pepper functions as a ribonuclease in an antiviral pathway. Plant J. 37, 186-198.

Parker, R., Flowers, T. J., Moore, A. L. and Harpham, N. V. (2006) An accurate and reproducible method for proteome profiling of the effects of salt stress in the rice leaf lamina. J. Exp. Bot. 57, 1109-1118.

Parry, M. A., Andralojc, P. J., Mitchell, R. A., Madgwick, P. J. and Keys, A. J. Manipulation of Rubisco: the amount, activity, function and regulation. J. Exp. Bot. 54, 1321-1333.

Pasternak D (1987) Salt tolerance and crop production—a comprehensive approach. Annu Rev Phytopathol 25: 271-291.

Pnueli, L., Hallak-Herr, E., Rozenberg, M., Cohen, M., Goloubinoff, P., Kaplan, A. and Mittler, R. (2002) Molecular and biochemical mechanisms associated with dormancy and drought tolerance in the desert legume Retama raetam. Plant J. 31, 319-330.

Portis, A. R. Jr. (2003) Rubisco activase—Rubisco's catalytic chaperone. Photosynth. Res. 75, 11-27.

Ramonell, K. M. and Somerville S. (2002) The genomics parade of defense responses: to infinity and beyond. Curr. Opin. Plant Biol. 5, 291-294.

Sachetto-Martins, G., Franco, L. O., de Oliveira and D. E. (2000) Plant glycine-rich proteins: a family or just proteins with a common motif? Biochim Biophys Acta. 1492, 1-14.

Salekdeh, G. H., Siopongco, J., Wade, L. J., Ghareyazie, B. and Bennett, J. (2002) Proteomic analysis of rice leaves during drought stress and recovery. Proteomics. 2, 1131-1145.

Salaita L, Kar R K, Manjee M, Downie A B (2005) Identification and characterization of mutants capable of rapid seed germination at 10° C. from activation-tagged lines of Arabidopsis thaliana. J Exp Bot 56: 2059-2069.

Sambrook and Russell (2001) Molecular Cloning: A Laboratory Manual, 3rd edition, Cold Spring Harbor Laboratory, Cold Spring Harbor (N.Y.)

Schardl, C., Byrd, A., Benzion, G., Altschuler, M., Hildebrand, D. and Hunt, A. (1987) Design and construction of a versatile system for the expression of foreign genes in plants. Gene, 61, 1-11.

Shafikhani S et al. (1997) Generation of large libraries of random mutants in Bacillus subtilis by PCR-based plasmid multimerization. Biotechniques 23(2):304-10.

Skriver, K. and Mundy, J. (1990) Gene expression in response to abscisic acid and osmotic stress. Plant Cell, 2, 503-512.

Somssich, I. E., Schmelzer, E., Kawalleck, P. and Hahlbrock, K. (1988) Gene structure and in situ transcript localization of pathogenesis-related protein 1 in parsley. Mol. Gen. Genet. 213, 93-98.

Srivastava, S., Fristensky, B. and Kav, N. N. V. (2004) Constitutive expression of a PR10 protein enhances the germination of Brassica napus under saline conditions. Plant Cell Physiol. 45, 1320-1324.

Srivastava S, Rahman M H, Shah S, Kav N N V (2006) Constitutive expression of pea ABA-responsive 17 (ABR17) cDNA confers multiple stress tolerance in Arabidopsis thaliana. Plant Biotech J 4: 529-549.

Stemmer W P (1994) DNA shuffling by random fragmentation and reassembly: in vitro recombination for molecular evolution Proc. Natl. Acad. Sci. USA 91(22), 10747-51.

Steppuhn H, Raney J P (2005) Emergence, height, yield of canola and barley grown in saline root zones. Can J Plant Sci 85: 815-827.

Steppuhn, J., Hermans, J., Nechushtai, R., Ljungberg, Ulf., Thummler, F., Lottspeich, F. and Herrmann, R. G. (1998) Photosynthesis is driven by two photosystems PSI and PSII in higher plants. FEBS Letters. 237, 218-224.

Stockinger E J, Gilmour S J, Thomashow M F (1997) Arabidopsis thaliana CBF1 encodes an AP2 domain-containing transcription activator that binds to the C-repeat/DRE, a cis-acting DNA regulatory element that stimulates transcription in response to low temperature and water deficit. Proc Natl Acad Sci USA 94: 1035-1040.

Subramanian, B., Bansal, V. K. and Kav, N. N. V. (2005) Proteome-level investigation of Brassica carinata-derived resistance to Leptosphaeria maculans. J. Agric. Food Chem. 53, 313-324.

Van Loon, L. C., Pierpoint, W. S., Boller, T. and Conejero, V. (1994) Recommendations for naming plant pathogenesis-related proteins. Plant Mol. Biol. Reporter, 12, 245-264.

Van Loon, L. C. and Van Strien, E. A. (1999) The families of pathogenesis-related proteins, their activities, and comparative analysis of PR-1 type proteins. Physiol. Mol. Plant Pathol. 55, 85-97.

Varotto, C., Pesaresi, P., Meurer, J., Oelmuller, R., Steiner-Lange, S., Salamini, F. and Leister, D. (2000) Disruption of the Arabidopsis photosystem I gene psaE1 affects photosynthesis and impairs growth. Plant J. 22, 115-124.

Vettakkorumakankav, N. N., Falk, D., Saxena, P. and Fletcher, R. A. (1999) A crucial role for gibberellins in stress protection of plants. Plant Cell Physiol. 40, 542-548.

Viera et al. (1987) Methods Enzymol. 153: 3.

Walter, M. H., Liu, J. W., Grand, C., Lamb, C. J. and Hess, D. (1990) Bean pathogenesis-related (PR) proteins deduced from elicitor-induced transcripts are members of a ubiquitous new class of proteins including pollen allergens. Mol. Gen. Genet. 222, 353-360.

Wang, W., Vinocur, B. and Altman, A. (2003) Plant responses to drought, salinity and extreme temperatures: towards genetic engineering for stress tolerance. Planta, 218, 1-14.

Wang, Z. Y. and Portis, A. R. Jr. (1992) Dissociation of Ribulose-1,5-Bisphosphate bound to Ribulose-1,5-Bisphosphate Carboxylase/Oxygenase and its enhancement by Ribulose-1,5-Bisphosphate Carboxylase/Oxygenase activase-mediated hydrolysis of ATP. Plant Physiol. 99, 1348-1353.

Wu, F., Li, Y. K., Chang, S., Zhou, Z., Wang, F., Song, X., Lin, Y. and Gong, W. (2003) Purification, characterization and preliminary crystallographic studies of a PR-10 protein from Pachyrrhizus erosus seeds. Acta Crystal. D. 58, 2165-2167.

Xu, D., Duan, X., Wang, B., Hong, B., Ho, T-H. D. and Wu, R. (1996) Expression of a late embryogenesis abundant protein gene, HVA1, from barley confers tolerance to water deficit and salt stress in transgenic rice. Plant Physiol. 110, 249-257.

Yamaguchi-Shinozaki K, Shinozaki K (1994) A novel cis-acting element in an Arabidopsis gene is involved in responsiveness to drought, low-temperature, or high-salt stress. Plant Cell 6: 251-264.

Yan, S., Tang, Z., Su, W. and Sun, W. (2005) Proteomic analysis of salt stress-responsive proteins in rice root. Proteomics. 5, 235-244.

Zaccolo M et al., (1996) An approach to random mutagenesis of DNA using mixtures of triphosphate derivatives of nucleoside analogues. J. Mol. Biol. 255(4), 589-603 (1996).

Zhang, H. X., Hodson, J. N., Williams, J. P. and Blumwald, E. (2001) Engineering salt-tolerant Brassica plants: characterization of yield and seed oil quality in transgenic plants with increased vacuolar sodium accumulation. Proc. Natl. Acad. Sci. USA, 98, 12832-12836.

Zhu, J-K. (2003) Regulation of ion homeostasis under salt stress. Curr. Opin. Plant Biol. 6, 441-445.

1. An expression vector for transformation of a plant cell comprising:
(a) a nucleic acid sequence encoding ABR17;
(b) regulatory elements operatively linked to the nucleic acid sequence such that the nucleic acid sequence encoding ABR17 is expressed in the plant cell,
wherein said expression results in expression of the ABR17 protein in said plant cell.

2. The vector of claim 1 wherein the nucleic acid sequence encodes the amino acid sequence of SEQ ID NO: 2.

3. The vector of claim 2 wherein the nucleic acid sequence comprises SEQ ID NO.1, or a portion or variant thereof.

4. A transgenic plant cell transformed with the expression vector of claim 1.

5. A transgenic plant seed comprising a transgenic plant cell of claim 4.

6. A transgenic plant grown from the transgenic plant cell of claim 4 or the transgenic plant seed of claim 5.

7. The transgenic plant of claim 6 which is a Arabidopsis sp. or a Brassica sp. plant.

8. A transgenic Arabidopsis plant of claim 7.

9. A transgenic Brassica sp. plant of claim 7.

10. A method of increasing the tolerance of a plant to at least one environmental stresses, comprising the steps of:
(a) transfecting cells of said plant with a nucleic acid sequence encoding ABR17,
(b) selecting and maintaining from said cells a transgenic cell line that expresses a protein encoded by said nucleic acid sequence, and
(c) producing a plant from the transgenic cell line;
(d) wherein the increased tolerance to the at least one environmental stress is demonstrated by one or more of enhanced germination, greater rate of flowering, earlier flowering, greater plant height, increased root length, increased shoot length, or overall plant health, as compared to a control plant.

11. The method of claim 10 wherein nucleic acid sequence encoding ABR17 encodes an amino acid sequence which comprises SEQ ID NO: 2.

12. The method of claim 11 wherein the nucleic acid sequence encoding ABR17 comprises SEQ ID NO:1, or a portion or variant thereof.

13. The method of claim 10, wherein the cells used in step (a) are from Arabidopsis sp.

14. The method of claim 10, wherein the cells used in step (a) are from Brassica sp.

15. A method of promoting early flowering in a plant, comprising the steps of:
(a) transfecting cells of said plant with a nucleic acid sequence encoding ABR17,
(b) selecting and maintaining from said cells a transgenic cell line that expresses a protein encoded by said polynucleotide, and
(c) producing a plant from the transgenic cell line.

16. The method of claim 15 wherein nucleic acid sequence encoding ABR17 encodes an amino acid sequence which comprises SEQ ID NO: 2.

17. The method of claim 16 wherein the nucleic acid sequence encoding ABR17 comprises SEQ ID NO:1.

18. The method of claim 15, wherein the cells used in step (a) are from Arabidopsis sp.

19. The method of claim 15, wherein the cells used in step (a) are from Brassica sp
